(19) United States
(12) Patent Application Publication
(10) Pub. No.: US 2011/0152880 A1
(43) Pub. Date: Jun. 23, 2011

(54) FLEXIBLE AND STEERABLE ELONGATE INSTRUMENTS WITH TORSION CONTROL

(75) Inventors: Jeffery B. ALVAREZ, San Mateo, CA (US); Enrique ROMO, Dublin, CA (US); Christopher CARLSON, Menlo Park, CA (US)

(73) Assignee: HANSEN MEDICAL, INC., Mountain View, CA (US)

(21) Appl. No.: 12/646,894

(22) Filed: Dec. 23, 2009

(51) Int. Cl. A61B 19/00

(52) U.S. Cl. 606/130

(57) ABSTRACT

An instrument for performing minimally invasive surgical procedures includes an elongate body and a support member disposed within or along the elongate body. The support member is configured to support steering, articulation, and angular rotational movement of the elongate body, provide torsion control, and support precise and accurate placement of the distal portion of the elongate body so that complex surgical procedure may be performed using the instrument.

### BACKGROUND

Standard surgical procedures or open surgeries typically involve using a scalpel to create an opening of sufficient size to allow a surgical team to gain access to an area in the body of a patient for the surgical team to diagnose and treat one or more target sites. When possible, minimally invasive surgical procedures may be used instead of standard surgical procedures to minimize physical trauma to the patient and reduce recovery time for the patient to recuperate from the surgical procedures. However, minimally invasive surgical procedures typically require using extension tools to approach and address the target site, and the typical extension tools may be difficult to use, manipulate, and control. Consequently, only a limited number of surgeons may have the necessary skills to proficiently manipulate and control the extension tools for performing complex minimally invasive surgical procedures. As such, standard surgical procedures or open surgery might be chosen for the patient even though minimally invasive surgical procedures may be more effective and beneficial for treating the patient. Accordingly, there is a need to develop extension tools that are easy to use, manipulate, and control, especially for performing complex minimally invasive surgical procedures.

### SUMMARY

Various embodiments described herein relate generally to robotically controlled systems, such as robotic or telerobotic surgical systems, and more particularly to flexible and steerable elongate instruments or catheters with sufficient stiffness and control to navigate and accurately place surgical instruments or tools in a precise manner on a target site for performing minimally invasive surgical procedures inside a patient.

In certain embodiments, systems or apparatus that may be configured for controlled steering and articulation of an elongate flexible member which maintain bending flexibility of the elongate flexible member while providing torsional stiffness along the length of the elongate flexible member are provided. The system or apparatus may be comprised of an elongate flexible member, means for steering, and means for torsional stiffness. Alternatively the disclosed system or apparatus may include passive elongate flexible members. The elongate member may be a catheter.

In accordance with one embodiment, an instrument for performing minimally invasive surgical procedures includes an elongate body and a support member disposed within the elongate body. The support member is configured to support steering and articulation movement of the elongate body and precise and accurate placement of the distal portion of the elongate instrument so that complex surgical procedure may be performed.

In accordance with one embodiment, the support member may provide substantial torsional stiffness along the length of the elongate body.

In one embodiment the torsional stiffness may be obtained by use of a tri-coil braided into the walls of a catheter. Each coil in the tri-coil may have a different cross sectional wire shape where, for example, the outer and inner coils may have flat rectangular wires while the middle coil may be comprised of round wires.

In another embodiment, each coil in the tri-coil may be wound from wires with substantially the same cross sectional shape but that cross sectional shape may be configured to allow interlocking with the axially adjacent winding of the wire coil. Thus each coil may allow for axial flexibility but the overlap in interlocked axially adjacent windings in that coil would close the spacing between windings causing the coil to function substantially as a compressible tube such that the radially adjacent coils may not overlap and herniate the elongate member.

Another embodiment may provide for each coil to be wound such that the distance between the windings varies down the length of the tri-coil so that bending can be maximized at some portions of the elongate member or catheter while minimized at other portions of the elongate member or catheter.

In other embodiments, a bi-coil construction using two coils wound in opposite directions may be used. Each bi-coil arrangement can also use variations of cross-sectional shapes for the wires and various spacing between the wires. The bi-coil may provide torsional stiffness in one rotational direction.

Other structural elements may be integrated into an elongate flexible member or catheter to provide structural support which can increase torsional stiffness. These elements may include ball and socket type joints and spacer-segment devices.

Additional elements may include flexible spines fabricated from tubular elements with patterns cut out so that the tubular element allows bending and compression but provides substantial torsional stiffness. A similar tubular element may be manufactured not from a single tube with laser cut patterns but from a series of interlocking elements, segments, or tubes coupled together to form an elongate flexible member. Alternatively additional mesh or braiding layers may be integrated into the elongate flexible member to increase torsional stiffness.

In certain embodiments, a flexible elongate body is provided. The flexible elongate body may include one or more axially extending members and one or more support members. The support members are configured to provide torsional stability to the flexible elongate body. The flexible elongate body may also include a base member, an end member and one or more intermediate spacer members.

Any of these elements and devices may be used by itself or in combination to provide the desired torsional stiffness or stability and bending flexibility in an elongate flexible member or catheter for a particular application. The different apparatuses disclosed may be used for passive elongate flexible members as well as steerable elongate members which can be robotically or non-robotically controlled.

In accordance with another embodiment, a method for performing a minimally invasive surgical procedure includes inserting an elongate instrument into a patient, e.g., through an incision, orifice or opening of an entry site. The elongate instrument includes a support member that allows at least one degree of freedom of movement of various portions of the elongate instrument. The method further includes advancing the elongate instrument along a pathway in the patient or through the entry site, steering and guiding a distal portion of the elongate instrument toward a target tissue structure through the pathway, and operating an instrument that is operatively coupled to the distal portion of the elongate instrument to diagnose or treat the target tissue structure.

In certain embodiments, a method of performing a minimally invasive diagnostic, surgical or therapeutic techniques is provided. The method may include inserting a flexible elongate body into a patient's body. The flexible elongate body may include one or more axially extending members and one or more support members. The support members may be configured to provide torsional stability to the flexible elongate body. The method may also include steering the elongate body from a first position to a second position in the body; transmitting torsion from a proximal end to a distal end of the elongate body with no or negligible or reduced torsion lag or wind-up while maintaining flexibility of the elongate body; and operating an instrument that is operatively coupled to a distal portion of the elongate body to diagnose or treat a target tissue structure in the body.

Other and further features and advantages of embodiments of the invention will become apparent from the following detailed description, when read in view of the accompanying figures.

### BRIEF DESCRIPTION OF THE DRAWINGS

The embodiments described herein will be readily understood by the following detailed description, taken in conjunction with accompanying drawings, illustrating by way of examples the principles of the invention. The objects and elements in the drawings are not necessarily drawn to scale, proportion, precise orientation or positional relationships; instead, emphasis is focused on illustrating the principles of the invention. The drawings illustrate the design and utility of various embodiments, in which like elements are referred to by like reference symbols or numerals. The drawings, however, depict the embodiments, and should not be taken as limiting their scope. With this understanding, various embodiments will be described and explained with specificity and detail through the use of the accompanying drawings in which:

FIG. 1 illustrates one example of a robotic or telerobotic surgical system.

FIG. 2 illustrates one example of a flexible and steerable elongate instrument with torsion control.

FIG. 3A through 3D illustrate other examples of flexible and steerable elongate instruments with torsion control.

FIG. 4A illustrates a single coil wound in a counter-clockwise direction.

FIG. 4B illustrates a single coil wound in a clockwise direction.

FIG. 4C illustrates a side view of a bi-coil with an inner coil wound in a counter-clockwise direction and an outer-coil wound in a clockwise direction.

FIG. 4D illustrates a top view of a bi-coil.

FIG. 5A illustrates a bi-coil with an inner coil wound in a counter-clockwise direction and an outer-coil wound in a clockwise direction.

FIG. 5B illustrates a single coil wound in a counter-clockwise direction.

FIG. 5C illustrates a side view tri-coil with an inner and outer coil wound in a counter-clockwise direction and a middle coil wound in a clockwise direction.

FIG. 5D illustrates a top view of a tri-coil.

FIG. 6A and FIG. 6B illustrate one embodiment of an elongate body of an elongate instrument or catheter that allows torsion control from a proximal portion of the elongate body to a distal portion of the elongate body.

FIG. 7A and FIG. 7B illustrate another embodiment of an elongate body of an elongate instrument or catheter that allows torsion control from a proximal portion of the elongate body to a distal portion of the elongate body.

FIG. 8A illustrates a cross sectional view of a catheter with an embedded bi-coil.

FIG. 8B illustrates a cross sectional view of a catheter with an embedded bi-coil in a bent configuration.

FIG. 8C illustrates a cross sectional view of a catheter with an embedded bi-coil in a bent configuration where an angular rotation is introduced at one end of the catheter.

FIG. 9A and FIG. 9B illustrate isometric views of one embodiment of the coils of a support member of an elongate body.

FIG. 10A illustrates a side and a sectional view of a single filar step coil.

FIGS. 10B and 10C each illustrate half of a sectional view of various embodiments of step shaped cross section wire forming a single filar step coil.

FIG. 11A illustrates a side and a sectional view of a single filar parallelogram coil.

FIGS. 11B and 11C each illustrate half of a sectional view of various embodiments of parallelogram shaped cross section wire forming a single filar parallelogram coil.

FIG. 12A illustrates a side and a sectional view of a double filar trapezoidal coil.

FIGS. 12B and 12C each illustrate half of a sectional view of various embodiments of trapezoidal shaped cross section wire forming a double filar trapezoidal coil.

FIG. 13A illustrates a side and a sectional view of a double filar t-shaped coil.

FIGS. 13B and 13C each illustrate half of a sectional view of various embodiments of t-shaped cross section wire forming a double filar t-shaped coil.

FIG. 14A through 14D illustrate cross sectional views of various tri-coils.

FIG. 14E illustrates a cross sectional view of a tri-coil in bending.

FIG. 14F illustrates a tri-coil catheter with an angular rotation applied at one end causing contraction of inner and outer coils and expansion of a middle coil.

FIG. 14G illustrates a tri-coil catheter with an angular rotation applied in the opposite direction causing expansion of inner and outer coils and contraction of a middle coil.

FIG. 15 illustrates a cross sectional view of one embodiment of a tri-coil support member.

FIG. 16A and FIG. 16B illustrate various embodiments of a “ball-and-socket” support member.

FIG. 17A through 17E illustrate various embodiments of a tubular support member.

FIG. 18A illustrates a front view of a “spacer-segment” support member.

FIG. 18B illustrates a top view of the “spacer-segment” support member of FIG. 18A.

FIG. 18C illustrates a side view of the “spacer-segment” support member of FIG. 18A.

FIG. 18D illustrates a top view of the “spacer-segment” of FIG. 18C.

FIG. 18E illustrates a side and top view of a spacer.

FIG. 18F illustrates a side and top view of a segment.

FIG. 18G illustrates an exploded side view of a “spacer-segment” support structure.

FIG. 19A-B illustrate variations of a dexterity device.

FIG. 19C illustrates an elongate instrument without torsional stiffness.

FIG. 19D illustrates an elongate instrument with a tri-coil added for torsional stiffness.

FIG. 19E illustrates an elongate instrument with mesh added for torsional stiffness.

FIG. 19F illustrates an elongate instrument with helical members added for torsional stiffness.

FIG. 20 illustrates a cross sectional view of one embodiment of a tri-coil support member.

### DETAILED DESCRIPTION OF ILLUSTRATED EMBODIMENTS

Reference will now be made in detail to embodiments of the present invention, examples of which are illustrated in the accompanying drawings. While the invention will be described in conjunction with the preferred embodiments, it will be understood that they are not intended to limit the scope of the invention to these embodiments. On the contrary, the invention is intended to cover alternatives, modifications, and equivalents that may be included within the spirit and scope of the invention. Furthermore, in the following detailed description of the present invention, numerous specific details are set forth in to order to provide a thorough understanding of the present invention. However, it will be readily apparent to one of ordinary skilled in the art that the present invention may be practiced without these specific details.

The contents of the following applications are incorporated herein by reference as though set forth in full for all purposes: U.S. patent application Ser. No. 11/073,363, filed on Mar. 4, 2005; U.S. patent application Ser. No. 11/418,398, filed on May 3, 2006; U.S. patent application Ser. No. 11/637,951, filed on Dec. 11, 2006; International Patent Application No. PCT/US2007/071535, filed on Jun. 19, 2007; U.S. patent application Ser. No. 12/079,500, filed on Mar. 26, 2008; U.S. patent application Ser. No. 12/126,814, filed on May 23, 2008; and U.S. patent application Ser. No. 12/242,196, filed on Sep. 30, 2008; and U.S. patent application Ser. No. 10/850,821, filed on May 21, 2004.

Standard minimally invasive surgical procedures commonly require the use of flexible steerable elongate members sometimes in the form of a catheter or guidewire which can be inserted through a small incision and then navigated through tortuous anatomy through an artery, natural body lumen, etc. In order to properly navigate to target anatomy, the catheter should have steerable control and also have substantial bending flexibility. Once in place, it is sometimes desirable for the elongate member to have the ability to either transmit rotational motion or resist rotational motion from its proximal end down along its length to its distal end in order to control the roll or rotation of extension tools that may be operatively coupled to the flexible and steerable elongate member at or near its distal tip. Additionally, providing resistance to torsion down the length of the catheter may allow better prediction of the roll position of the catheter distal tip allowing for better catheter control.

Simaan et al. (U.S. Patent Application No. 2005/0059960) describes a manipulation device in the form of an elongate steerable member that includes a series of disks separated by a plurality of tubular backbones. The backbones are substantially flexible in bending but substantially axially stiff so they may be used in a push and pull manner to steer the elongate member. While this apparatus allows for steering and bending flexibility, the construction of this flexible member does not provide for any torsional resistance so it can neither resist nor transmit torsion.

Currently, different types of apparatus can be used to transmit torsion down the length of an elongate flexible member. Cables with a tri-coil configuration are often used in bicycles and automobiles. The tri-coil may be constructed of 3 different wires that may be helically wound together in opposing directions. The result may be an elongate flexible member comprised of an inner, middle and outer coil with the outer and inner wires that may be wound in one direction while a middle wire may be wound in the opposite direction. As one end of the cable is rotated, each individual coil may have a tendency to move in the radial direction, expanding or contracting in diameter depending on the direction it is rotated much like a constrained spring would. Because the three coils are wound together in opposite directions, the middle coil may expand while the outer and inner coils may contract and vice versa depending on the direction of rotation or winding of the coils. The opposing resistance between the coils to either expand or contract prevents the overall tri-coil combination from changing in diameter. Instead the tri-coil will transmit the rotation imparted on the proximal end to the distal tip of the elongate member.

Attempts have been made to incorporate this same tri-coil configuration into a catheter to increase its overall torsional stiffness. However, because catheters require bending flexibility, the coils must be wound such that there is spacing between axially adjacent windings of a coil allowing for axial compression and expansion. As the catheter bends, the spacing between the windings of a coil will decrease at the inner bend. Smaller spacing between the windings of a coil will decrease the axial compression and thus decrease the bending flexibility of the catheter. If the coils are wound with the larger spacing needed to achieve the required bending flexibility, the three coils may lose their resistance to radial expansion or contraction, moving into the spacing between windings of radially adjacent coils. They may begin to overlap radially causing the catheter to herniate. Ideally, the coils should be configured to have flexibility in the axial direction while being constrained in the radial direction. Certain embodiments provide torsional stiffness to a flexible elongate member while still preserving bending flexibility.

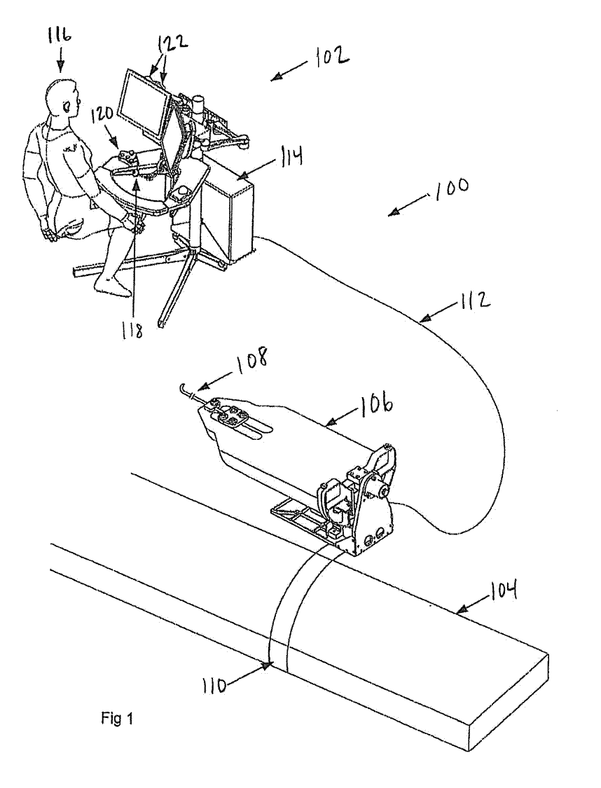

All of the following described technologies may be utilized or compatible with manually or robotically steerable instruments, such as those described in the aforementioned patent applications. FIG. 1 illustrates one example of a robotic or telerobotic surgical system (100), e.g., the Sensei® Robotic Catheter System from Hansen Medical, Inc. in Mountain View, Calif., U.S.A., with an operator control station (102) located remotely from an operating table (104) to which an electromechanical device, instrument driver, or robotic catheter manipulator (RCM) (106) and instrument assembly or steerable catheter assembly (108), e.g., the Artisan™ Control Catheter also from Hansen Medical, Inc. in Mountain View, Calif., U.S.A., may be supported by an instrument driver mounting brace (110) that is mounted on the operation table (104). A wired connection (112) transfers signals between an electronics rack (114) located near the operator control station (102) and the instrument driver (106) mounted near the operation table (104). The electronics rack (114) includes system hardware and software that operate and perform the many functions of the robotic or telerobotic surgical system (100). The instrument driver mounting brace (110) may be a substantially arcuate-shaped structural member configured to position the instrument driver (106) above a patient (not shown) who is lying on the operating table (104). The wired connection (112) may transmit manipulation, articulation, and control commands from an operator or surgeon (116) who is working at the operator control station (102) and who may be providing the necessary input to the instrument driver (106) by way of one or more input devices, such as an instinctive Motion™ controller (118), joystick, keyboard (120), trackball, data gloves, exoskeletal gloves, or the like, for operating the instrument assembly (108) to perform various operations, such as minimally invasive procedures, on the patient who is lying on the operating table (104). The wired connection (112) may also transmit information (e.g., visual, tactile, force feedback, position, orientation, shape, localization, electrocardiogram, etc.) from the instrument assembly (108), patient, and operation site monitors (not shown in this figure) to the operator control station (102) for providing the necessary information to the operator or surgeon (116) to facilitate monitoring the instruments, patient, and target site for performing various precise manipulation and control of the instrument assembly (108) during minimally invasive surgical procedures. The wired connection (112) may be a hard wire connection, such as an electrical wire configured to transmit electrical signals (e.g., digital signals, analog signals, etc.), an optical fiber configured to transmit optical signals, a wireless link connection configured to transmit various types of wireless signals (e.g., RF signals, microwave signals, etc.), etc., or any combinations of electrical wire, optical fiber, and/or wireless links. The wire connection (112) allows the surgeon or operator (116) to be remotely located from the patient. The surgeon or operator (116) may be located across the operation room from the patient, in a different room, in a different building, or in a different geographical region away from where the patient is located. Information or feedback transmitted by way of the wire connection (112) may be displayed on one or more monitors (122) at the operator control station (102).

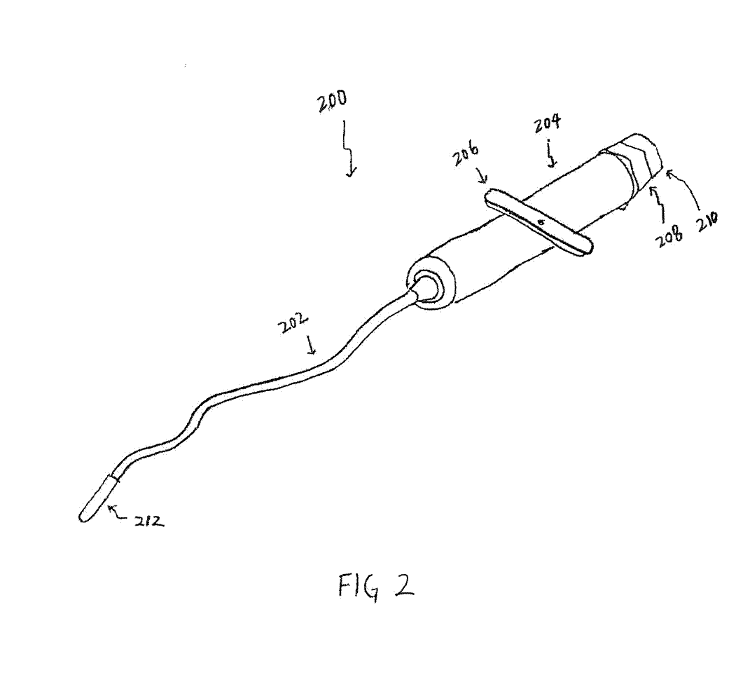

FIG. 2 illustrates a flexible and steerable elongate instrument in accordance with one embodiment, which may be used as an extension tool to deliver and/or operate various surgical instruments or tools to a target site for performing various minimally invasive surgical procedures inside a patient. As illustrated in FIG. 2, the flexible elongate instrument (200) includes an elongate body (202) that may be manually pushed, advanced, steered, rotated, and maneuvered inside the pathways of a patient toward a target site. The elongate body (202) may be substantially stiff (e.g., rotationally stiff) so as to allow transfer of rotational motion or torque from a proximal end of the elongate instrument to a distal end of the elongate instrument without significant torsion lag or wind up. The elongate body (202) may have an outer diameter in the range of about 1.5 French to about 20 French—in the French catheter measurement scale. In some embodiments, the elongate body (202) may have an outer diameter of about 11 French or about 12 French. In certain other applications, the elongate body (202) may have an outer diameter of about 9 French, about 8 French, about 7 French, or about 6 French.

In this example, the elongate instrument (200) may include a handle (204) and a control lever (206) to allow manual operation of one or more control wires or pull wires to steer the distal portion of the elongate body (202) as the elongate body is pushed or advanced through various tortuous pathways toward a target site inside a patient. In other embodiments, the elongate instrument (202) may be robotically operated or controlled. The use of control wires or pull wires to steer an elongate body has been described in connection with various manually or robotically operated systems. Examples of such steerable systems are disclosed in U.S. patent application Ser. No. 11/073,363, titled “Robotic Catheter System”, filed on Mar. 4, 2005; and U.S. patent application Ser. No. 11/481,433, titled “Robotic Catheter System and Methods”, filed on Jul. 3, 2006. In addition, a first control knob (208) and a second control knob (210) may be manually operated to rotate elements or components of the elongate body (202), such that rotation or torque applied at the first control knob (208) and/or second control knob (210), either separately or in concert, transmits rotation or torque or torsion from the proximal portion of the elongate body (202) to the distal portion of the elongate body (202). In some embodiments, the elongate body (202) may also include a through lumen such that surgical instruments or tools may be delivered or advanced from a proximal portion of the elongate body (202) to a distal portion of the elongate body (202), such that the surgical instrument or tools may be placed and operated at a target site inside the body of a patient. In some embodiments, instead of delivering or advancing surgical instruments or tools from the proximal portion of the elongate body (202) to the distal portion of the elongate body (202), the surgical instruments or tools may be operably mounted or coupled to the distal portion of the elongate (202).

As will be discussed in more detail, the elongate body (202) and elements of the elongate body (202) may be designed and manufactured to be substantially stiff for torsional applications, such that there may be a minimum or reduced amount of torque or torsion deflection or torque or torsion lag from one section (e.g., proximal section) to another section (e.g., distal section) of the elongate body. In this manner, movement or motion control input provided at the proximal portion of the elongate body (202) may result in accurate and predictable movement or motion output at the distal portion of the elongate body (202). The elongate body (202) or elements of the elongate body (202) may also be designed and manufactured to be substantially flexible, so that the elongate body (202) may be steered, maneuvered, or deflected in various directions (e.g., up, down, pitch, yaw, etc.) as well as bent or displaced into various positions, shapes, and/or complex curvatures (e.g., J-bend or J-shaped bend).

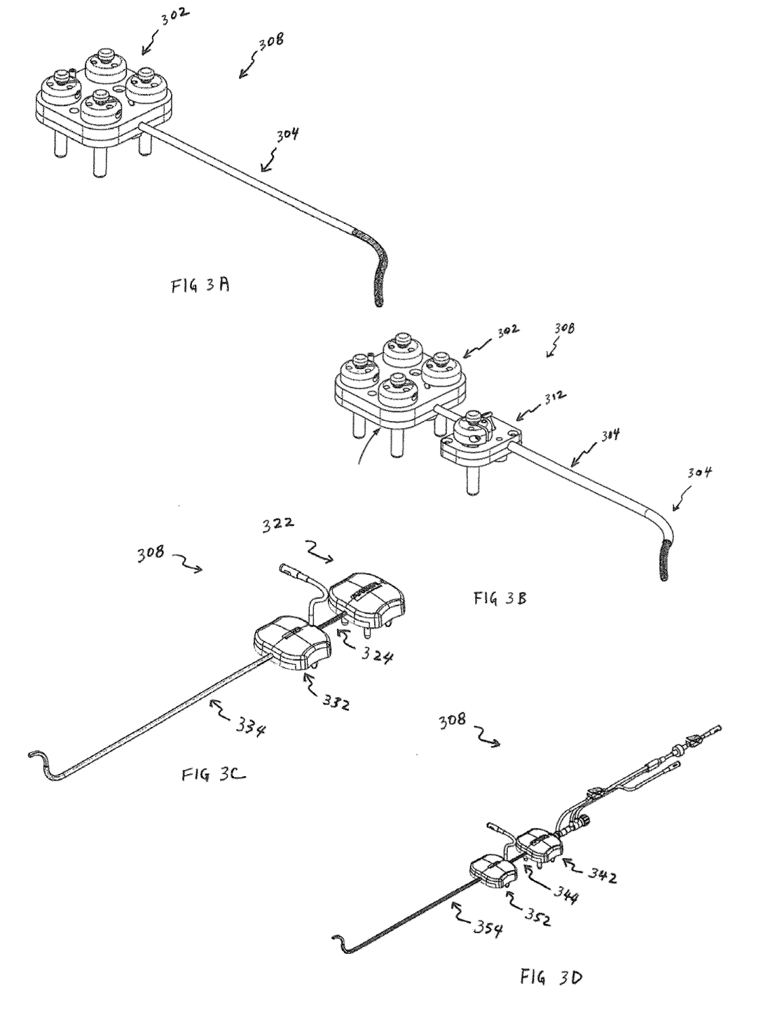

FIG. 3A through FIG. 3D illustrate various embodiments of robotically operated elongate instrument assemblies (308) that may be configured to deliver and/or operate various surgical instruments or tools for performing minimally invasive surgical procedures inside a patient. An elongate instrument assembly (308) may be comprised of a single steerable elongate instrument assembly or catheter system, as illustrated in FIG. 3A, or a combination of steerable elongate instrument assemblies or catheter systems, as illustrated in FIG. 3B through 3D. As illustrated in FIG. 3B through 3D, the steerable elongate instrument assemblies or catheter systems may be positioned or mounted in a substantially coaxial manner and configured to be operated in a substantially coordinated or tandem manner or as a coordinated or tandem combination. As described in the aforementioned patent applications that have been incorporated by reference, the instrument assembly (308) may include a control unit or splayer; which may be comprised of gears, pulleys, and control or pull wires to steer or articulate an elongate instrument or catheter in various degrees of motion (e.g., up, down, pitch, yaw, or any motion in-between as well as any other motions). For example, FIG. 3A illustrates one embodiment of an instrument assembly or catheter system (308) which includes a control unit (302) that may be configured to steer an elongate instrument or catheter (304). FIG. 3B illustrates another embodiment of an instrument assembly (308) that includes a combination of steerable elongate instrument assemblies or catheter systems which includes respective control units (302 and 312) and corresponding associated elongate instruments or catheters (304 and 314). The elongate instrument assemblies or catheter systems, as those illustrated in FIG. 3B as well as other similar systems or combinations, may be positioned or mounted coaxially with the elongate instrument or catheter of one elongate instrument assembly or catheter system threaded or loaded through a lumen of another elongate instrument assembly or catheter system. FIG. 3C also illustrates an instrument assembly (308) that includes a combination of steerable elongate instrument assemblies or catheter systems which are comprised of respective control units or splayers (322 and 332) and corresponding associated elongate instruments or catheters (324 and 334). FIG. 3D illustrates another embodiment of an instrument assembly (308) that includes a combination of steerable elongate instrument assemblies and catheter systems which may also include respective control units or splayers (342 and 352) and corresponding associated elongate instruments or catheters (344 and 354).

For each embodiment of an elongate body, elongate instrument assembly, or catheter as previously described, the elongate body or elongate instrument may be controlled in a “roll degree” of freedom. Roll control may be accomplished by rotating the proximal end of the elongate body or elongate instrument. If the elongate body or elongate instrument is not torsionally stiff, the elongate body may twist or wind up when the proximal end is rotated. Thus the angular rotation at the distal portion or tip of the elongate body may not accurately match the angular rotation at the proximal end. A loss of control and predictability of the elongate body rotation or position may be experienced as well as a loss of control of any extension tools that may be operatively coupled to the distal portion of the elongate body that require rotational control.

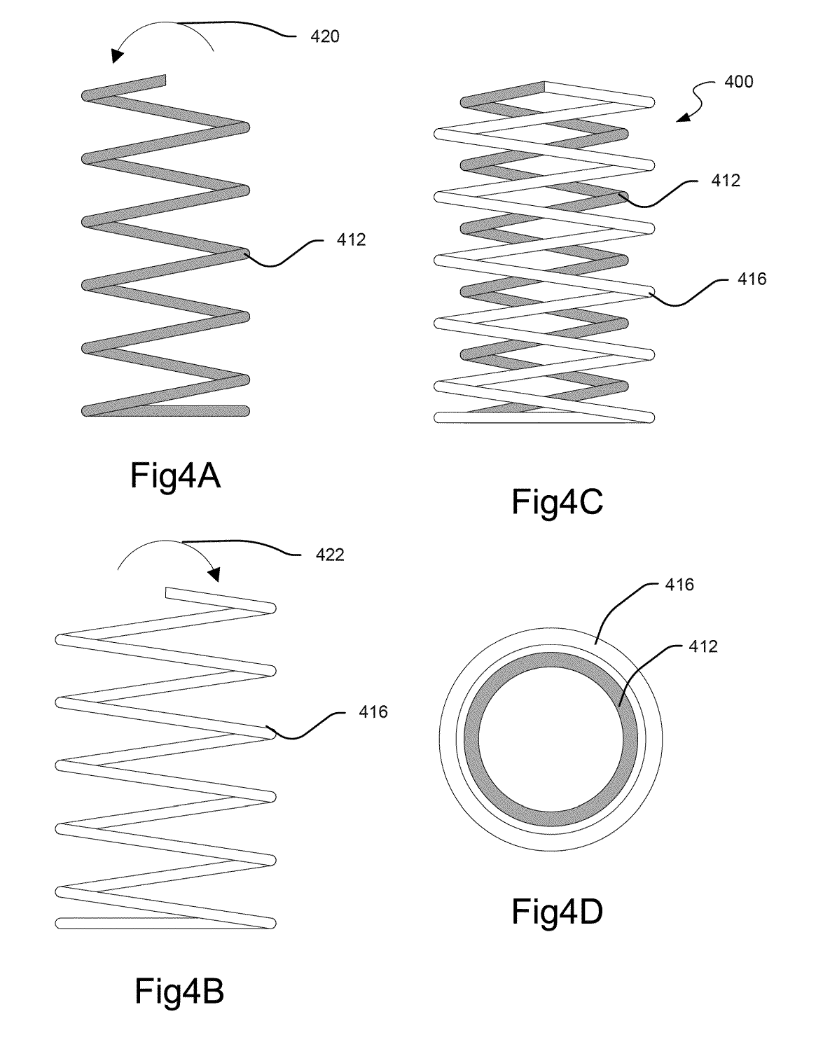

An elongate flexible member, e.g., an elongate body, elongate instrument or catheter may include a support member for providing torsional stability or stiffness to the elongate member. Various support members are contemplated herein. In certain embodiments, a bi-coil support member may be braided, embedded or otherwise coupled to the elongate body in order to increase the elongate body torsional stiffness. FIGS. 4A-4D illustrate an embodiment of a bi-coil. The bi-coil (400) may be comprised of an inner coil (412) and an outer coil (416) which may be helically wound in opposite directions from one another. FIG. 4A illustrates an inner coil (412) may be wound in a counter clockwise direction (420) while FIG. 4B illustrates an outer coil (416) that may be wound in a clockwise direction (422). The outer coil (416) may have a larger coil diameter than the inner coil (412) which may surround the inner coil (412) as illustrated in FIG. 4C to create a bi-coil (400) combination, element, or support member. FIG. 4D illustrates a top view of the bi-coil member (400). In an alternate embodiment, the inner coil may be wound in the clockwise direction and the outer coil may be wound in the counter clockwise direction.

A bi-coil support member may provide torsional stiffness in one rotational direction (e.g., clockwise or counter-clockwise direction), depending on the directions of the windings of each coil in the following manner. As each single coil is rotated in the same direction that its wire is wound, if there is any constraint of motion along the body or at the distal tip of the coil, the coil will have a tendency to decrease in diameter or contract. That same coil rotated in the opposite direction that its wire is wound will have a tendency to increase in diameter or expand. An inner and outer coil wound in opposite directions will expand and contract respectively in one rotational direction interfering or opposing with one another. This interference or opposing action will create torsional stiffness or induced torsional stiffness in that rotational direction (e.g., clockwise or counter-clockwise direction). Because each coil is not able to expand or contract, the rotation at the proximal end of the bi-coil will be transmitted down the length of the bi-coil without substantial wind up. In the opposite rotational direction, however, the inner coil will contract while the outer coil will expand, as such no interference or opposing expansion or contraction movements of the coils will result. Thus, there will be no torsional stiffness or induced torsional stiffness in the opposite rotational direction in the bi-coil configuration.

In certain embodiments, a flexible elongate body may include first and second helical members or coils which are configured such that when a rotational force is applied to the flexible elongate body the first and second helical members are driven in opposing radial directions interfering with one another in opposing radial directions. Also, at least a first helical member may include overlaying or interlaying between axially adjacent windings of the first helical member which acts to minimize or prevent overlap between radially adjacent windings of the first helical member and a second helical member to provide torsional stability to the flexible elongate body.

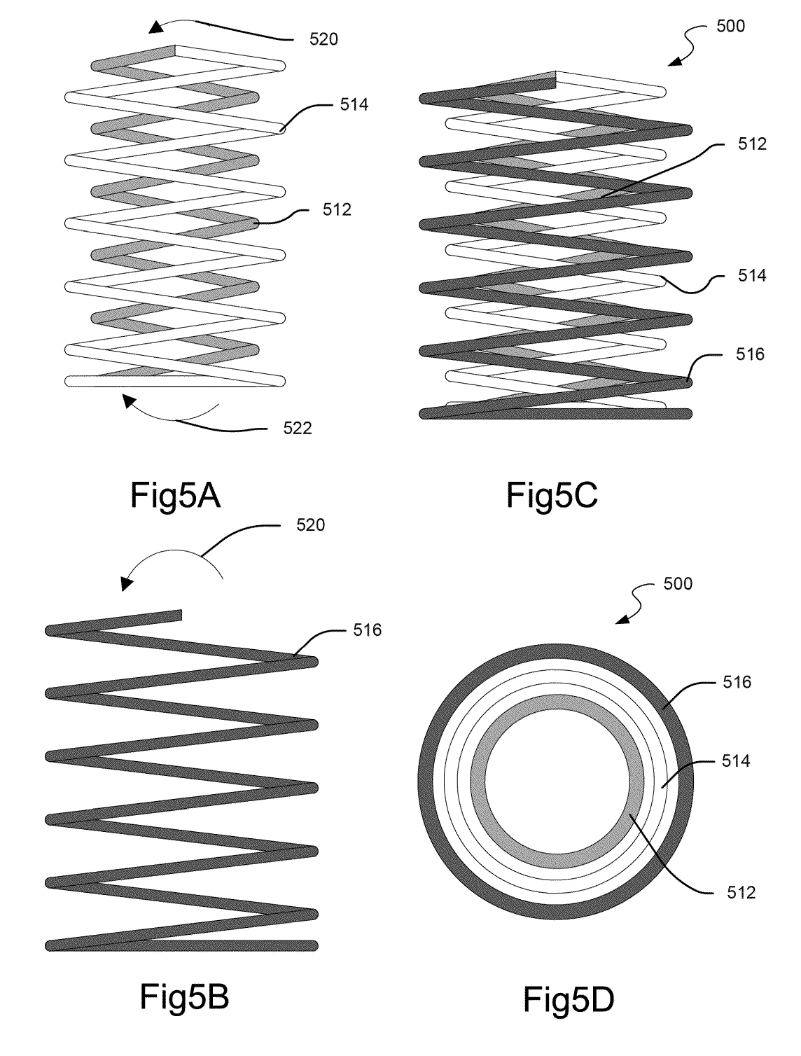

In order to achieve torsional stiffness or induced torsional stiffness in both rotational directions (e.g., clockwise and counter clockwise directions), a tri-coil support member may be embedded, braided, or otherwise coupled to an elongate body. A tri-coil (500) may be comprised of an inner (512), middle (514) and outer coil (516) as shown in FIGS. 5A-5D. The inner (512) and outer (516) coil may be wound in one direction (520) while the middle coil may be wound in the opposite direction (522). FIG. 5D illustrates a top view of the tri-coil (500). In a manner substantially similar to how a bi-coil functions, a tri-coil will effectively provide torsional stiffness or induced torsional stiffness in both rotational directions from interference or opposing expansion and contraction movements of the tri-coils.

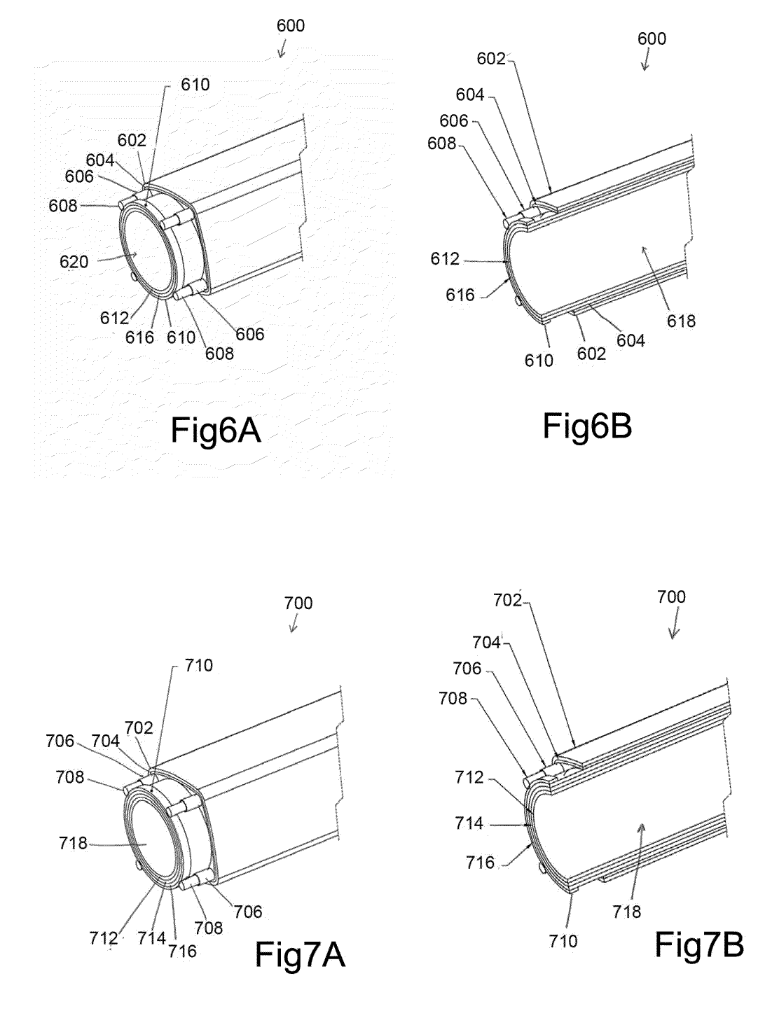

FIG. 6A and FIG. 6B illustrate one embodiment of an elongate body (600) of an elongate instrument or catheter in which a bi-coil may be braided, embedded or otherwise coupled to the elongate body in order to increase the elongate body torsional stiffness. The elongate body (600) may be substantially flexible in various bending modes such that it may be easily steered, bent, or deflected in various directions (e.g., up, down, side-to-side, pitch, yaw, etc.) as well as into various shapes or curvatures (e.g., J-shape, S-shape, etc.). In addition, the elongate body (600) may be substantially stiff in rotational mode, such that it may be able to resist or support angular rotation. The elongate body may be able to resist as well as support and/or transmit rotation or torque or torsion with no or substantially negligible or a reduced amount of torque or torsion lag or wind up from a proximal end of the elongate body to a distal end of the elongate body. In this manner, movement or motion control input provided at the proximal portion of the elongate body (600) may result in accurate, precise, and predictable movement or motion output at the distal portion of the elongate body (600).

As illustrated in FIG. 6A, the elongate body (600) includes an outer cover, outer layer, or outer jacket (602), a middle cover or middle layer (604), an inner cover or inner layer (606), pull wires or control wires (608), a retainer or retaining ring (610), an outer coil (616) and an inner coil (612). In this example, the elongate body (600) may be considered as a bi-coil structure where the inner coil may be wound in a direction opposite the outer coil. The coil construction allows the elongate body to be substantially flexible for some movements or applications of the elongate body, while substantially stiff for some other movements or applications. In other words, the bi-coil core construction with two coils allows the elongate body to be flexibly steered, bent, or deflected in various directions (e.g., up, down, side-to-side, pitch, yaw, etc.) as well as into various shapes or curvatures (e.g., J-shape, S-shape, etc.), while substantially stiff in rotation. That is, the bi-coil construction allows the elongate body to support and/or transmit rotation and torque or torsion with no or negligible or a reduced amount of torque or torsion lag or wind up from the proximal end of the elongate body to a distal end of the elongate body in one rotational direction (e.g., clockwise or counter-clockwise direction) but not the opposite direction in the manner described previously for bi-coils. The rotational stiffness property or characteristic of the elongate body allows for accurate, precise, and predictable movement of the elongate body. The middle layer (604) may be a braided layer that wraps around the coil core of the elongate body (600). The middle layer (604) may provide some structural support to the coil core of the elongate body (600) without substantially affecting the flexibility or movements of the elongate body (600). The inner layers or inner covers (606) secure the pull wires or control wires (608) to the elongate body. The inner layers or inner covers (606) may be a polyimide material. The pull wires (608) may be operated to steer or navigate the elongate body (600) in various directions. The pull wires (608) may have a round or substantially flat or rectangular-like cross section. The pull wires (608) may be wires or threads and they may be made of metallic, polymeric, synthetic, or natural materials. The retainer or retaining ring (610) may be used to secure the outer coil (616) and inner coil (612). Within the elongate body (600), a lumen (618) provides a passage way or channel in which tools or instruments may be advanced from the proximal portion of the elongate body to the distal portion of the elongate body. FIG. 6B illustrates a cross sectional view of the elongate body (600) in which various components of the elongate body may be more easily discerned.

FIG. 7A and FIG. 7B illustrate another embodiment of an elongate body (700) of an elongate instrument or catheter. In order to achieve torsional stiffness or induced torsional stiffness in both rotational directions (e.g., clockwise and counter-clockwise directions), a tri-coil can be embedded, braided, or otherwise coupled to an elongate body. The elongate body (700) may be substantially flexible in various bending modes such that it may be easily steered, bent, or deflected in various directions (e.g., up, down, side-to-side, pitch, yaw, etc.) as well as into various shapes or curvatures (e.g., J-shape, S-shape, etc.). In addition, the elongate body (700) may be substantially stiff in rotational mode, such that it may be able to resist or support angular rotation. The elongate body may be able to resist as well as support and/or transmit rotation or torque or torsion with no or negligible or a reduced amount of torque or torsion lag or wind up from a proximal end of the elongate body to a distal end of the elongate body. The rotational stiffness property or characteristic of the elongate body allows for accurate, precise, and predictable movement of the elongate body.

As illustrated in FIG. 7A, the elongate body (700) includes an outer cover, outer layer, or outer jacket (702), a middle cover or middle layer (704), an inner cover or inner layer (706), pull wires or control wires (708), a retainer or retaining ring (710), an outer coil (716), a middle coil (714) and an inner coil (712). In this example, the elongate body (700) may be considered as a tri-coil structure. The coil construction allows the elongate body to be substantially flexible for some movements or applications of the elongate body, while substantially stiff for some other movements or applications. That is, the coil core construction with one or more coils allow the elongate body to be flexibly steered, bent, or deflected in various directions (e.g., up, down, side-to-side, pitch, yaw, etc.) as well as into various shapes or curvatures (e.g., J-shape, S-shape, etc.), while at the same time being substantially stiff in rotation. The coil construction allows the elongate body to support and/or transmit rotation, torque, torsion, or twist with no or substantially negligible or a reduced amount of torque or torsion lag or wind up from the proximal end of the elongate body to a distal end of the elongate body in various rotational directions (e.g., clockwise and counter-clockwise directions) in the manner described previously for tri-coils. The middle layer (704) may be a braided layer that wraps around the coil core of the elongate body (700). The middle layer (704) may provide some structural support to the coil core of the elongate body (700) without substantially affecting the flexibility or movements of the elongate body (700). The inner layers or inner covers (706) secure the pull wires or control wires (708) to the elongate body. The pull wires (708) may be operated to steer or navigate the elongate body (700) in various directions. The retainer or retaining ring (710) may be used to secure the outer coil (716), middle coil (714) and inner coil (712). Within the elongate body (700), a lumen (718) provides a passage way or channel in which tools or instruments may be advanced from the proximal portion of the elongate body to the distal portion of the elongate body. The surgical instruments or tools may be placed at precise locations of a target site inside the body of a patient for diagnostic or treatment procedures. FIG. 7B illustrates a cross sectional view of the elongate body (700) in which various components of the elongate body may be more easily discerned.

As discussed in the examples illustrated in FIG. 6A, FIG. 6B, FIG. 7A and FIG. 7B, the coil structures may be the main structural support elements of the elongate body. The coil structure may provide torsion transmission without significant torque or torsion lag or wind up while allowing for axial compression or deflection so the elongate body may be substantially flexible for driving, steering, or navigating in various directions or form various complex shapes. In order to achieve flexibility in bending, the coils must allow for axial compression which may be achieved by increasing spacing or gaps between axially adjacent windings of each coil. Larger spacing or gaps may allow for greater axial compression and smaller bend radii of the elongate body which may allow for greater bending flexibility. However as spacing between axially adjacent windings increases, each coil tends to expand or collapse into the spacing of the radially adjacent coil. The radially adjacent windings of the coils may overlap with each other and herniate the catheter.

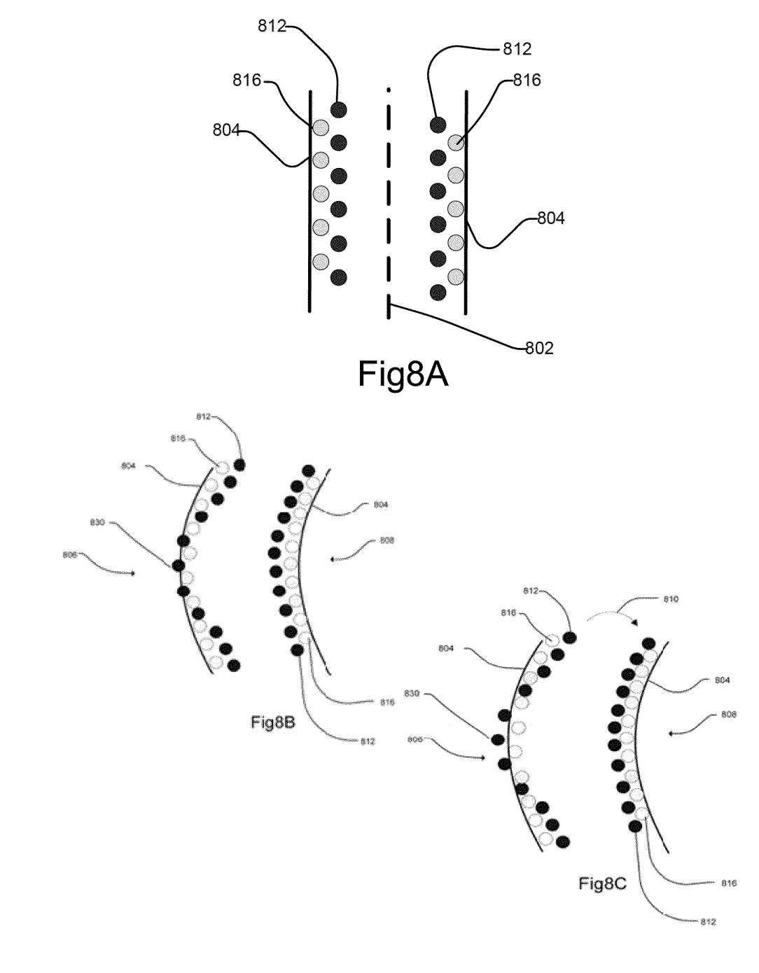

FIG. 8A shows a cross section of a catheter (800) with an integrated bi-coil comprised of an inner coil (812) and an outer coil (816) in a straight configuration. The neutral axis (802) of the catheter is also shown as the central axis of the catheter wall (804). FIG. 8B shows the catheter (800) in a bent configuration. As the catheter bends, the spacing between axially adjacent windings or coils on the outer bend (806) of each of the coils increases while the spacing between axially adjacent windings or coils on the inner bend (808) of each of the coils decreases. The windings or coils of the coils on the outer bend also tend to expand outward. As a result, on the outer bend (806) the spacing between axially adjacent windings or coils of the outer coil (816) allows for windings or coils of the inner coil (812) to protrude through or overlap with the outer coil (816) creating a hernia (830) of the catheter wall. FIG. 8C shows the catheter in a bent configuration when an angular rotation (810) is introduced. In this example, the inner coil (812) expands radially and the outer coil (816) contracts radially. On the inner bend, the two coils (812,816) interfere with one another and prevent further expansion. However, on the outer bend (806) the spacing between axially adjacent windings or coils of the outer coil (816) allows for a few windings or coils of the inner coil (812) to protrude through or overlap with the outer coil (816) creating a hernia (830) of the catheter wall. Either bending or applied rotation or a combination of bending and applied rotation can cause this hernia to be created in the catheter wall depending on the configuration of the catheter with bi-coil. This phenomenon can also occur in a similar manner with a tri-coil configuration.

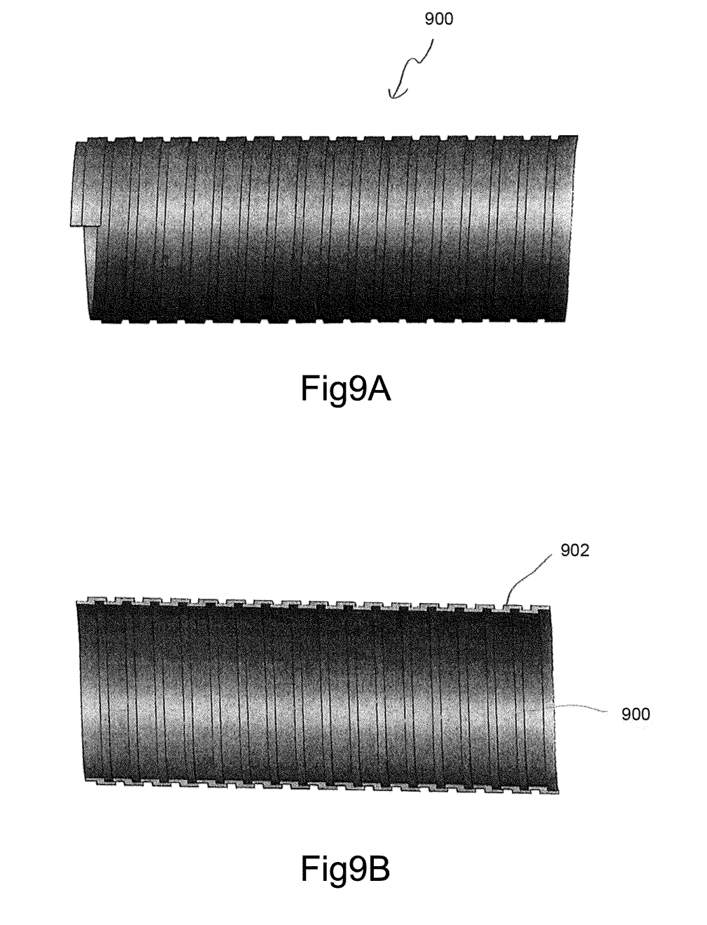

In order to allow for axial compressibility while still preventing the coils from overlapping, the cross-section of each wire in each coil may be such a shape that allows for axial movement while preventing overlap of radially adjacent windings. FIG. 9A and FIG. 9B illustrate side views of a coil structure (900) and they illustrate how the features of a coil winding may overlay or interlay with axially adjacent coil windings (902). By using a wire with a certain cross sectional shape, the axially adjacent windings may link together to essentially form a solid tube (900). The cross sectional shape of the wire; however, could be one that allows for axial compression and expansion. Thus, when three coils alternately wound in opposite directions are used within a tri-coil construction, for example, the tri-coil may provide for torsional stiffness with substantial axial expansion/compressibility for bending, but because each coil acts substantially as a tubular structure, the windings in radially adjacent coils may not overlap and the coils may not herniate.

Different cross sectional wire shapes additionally lend themselves to be wound in a multi-filar construction, a technique involving winding several adjacent wires together into a single coil which is well known in the art for creating coils. Various embodiments comprised of various cross sectional shapes and various filar constructions will be herein described.

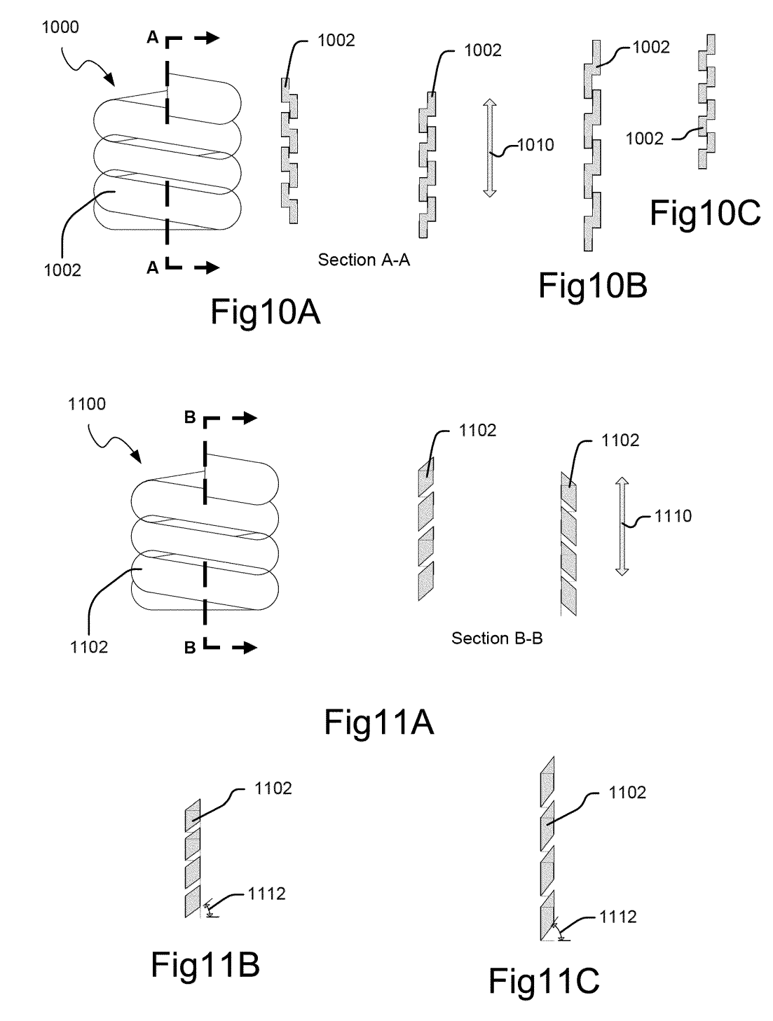

In one embodiment, the cross section of the wires in each coil may be a step shape configuration as shown in FIG. 10A. FIG. 10A illustrates a side and a sectional view of single filar step coil (1000) wound from a step shaped wire (1002). In this embodiment, a single wire can be wound to create the coil which may be flexible in the axial direction (1010) allowing for compression and extension of the coil. However, as the coil expands, the steps continue to overlap such that the coil acts like a tube with axial compressibility. To allow for greater expansion or compression the step shape may not be limited to the dimensional scale illustrated in FIG. 10A. In other embodiments, the step shape may be elongated to allow for more axial extension of the coil as illustrated in FIG. 10B or the central member on the step may be decreased in width to allow for more axial compression as illustrated in FIG. 10C.

In an alternative embodiment the cross section of the wire may be a parallelogram as illustrated in FIG. 11A. FIG. 11A illustrates a side and a sectional view of single filar parallelogram coil (1100) wound from a parallelogram shaped wire (1102). In this embodiment, a single parallelogram shaped wire (1102) may be wrapped helically to create the single filar parallelogram coil (1100) that allows for axial compression and extension (1110) while preventing radial expansion and contraction. FIGS. 11B and 11C illustrate various alternatives to the dimensions of the parallelogram cross section. By increasing or decreasing the angle (1112) of the sides of the parallelogram as shown in FIG. 11B, the amount of compression or expansion may be altered. A smaller value of the angle (1112) may provide for less axial expansion/compression as illustrated in FIG. 11B while FIG. 11C illustrates a larger value for (1112) which may allow for more axial expansion/compression.

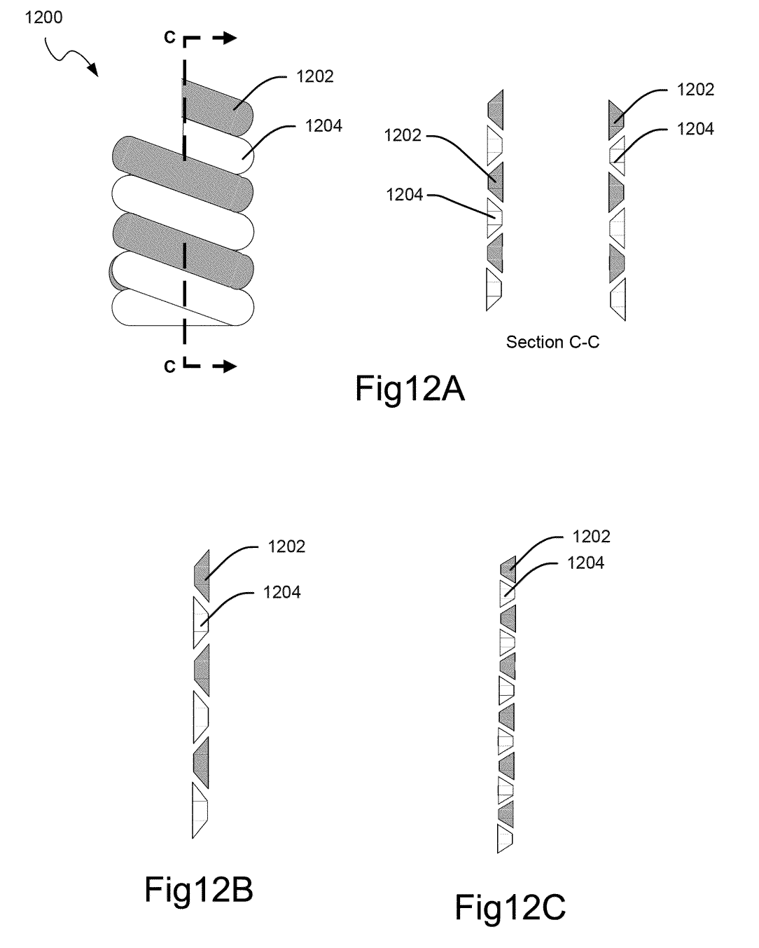

FIG. 12A illustrates another embodiment wherein the cross section of the wire in each coil may be a trapezoidal shape. With a trapezoidal cross wire shape, a double filar construction should be used in which two wires are adjacently wound into one coil double filar trapezoidal coil (1200). During construction, wire 1 (1202) would be placed adjacent to but mirrored with wire 2 (1204) and the two wires would be wound into the double filar trapezoidal coil (1200). The trapezoidal coil would allow for axial flexibility and create radially solid construction in the same manner as previously mentioned shape configurations. Various dimensions for the trapezoid can increase the axial expansion/compression as illustrated in FIG. 12B or decrease the axial expansion/compression as illustrated in FIG. 12C.

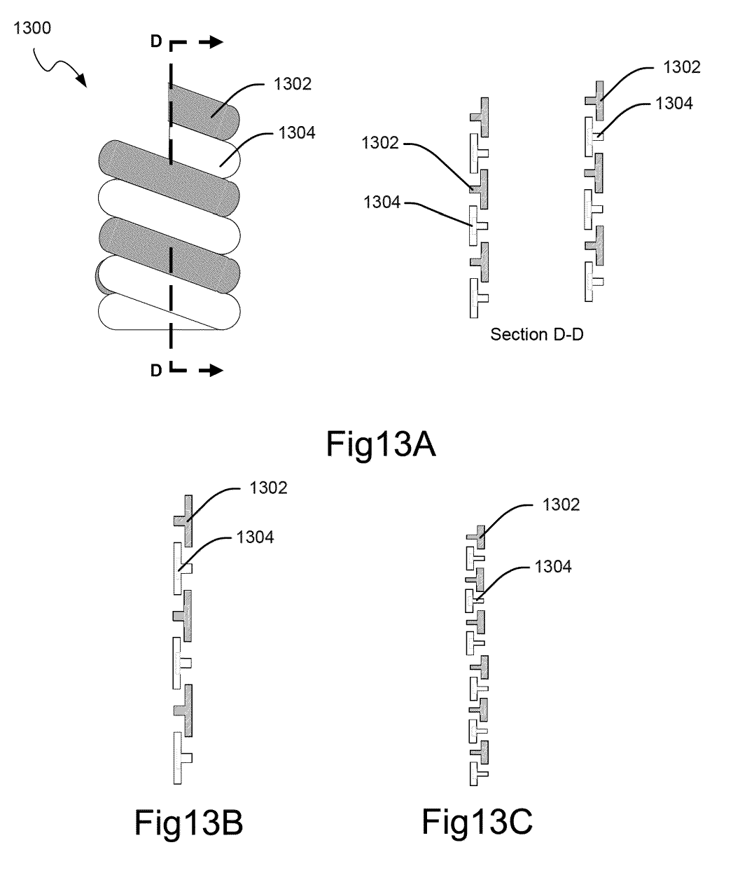

FIG. 13A illustrates another embodiment where the cross section of the wires in each coil may be a T-shape configuration. With a T-shaped cross wire, a double filar construction should be used in which two wires are adjacently wound into one double filar t-shaped coil (1300). During construction wire 3 (1302) would be placed adjacent to but mirrored with wire 4 (1304) and the two wires would be wound into the double filar t-shaped coil (1300). The t-shaped coil would allow for axial flexibility and create the radially constrained construction in the same manner as previously mentioned shape configurations. Various dimensions for the t-shape can increase the axial expansion/compression as illustrated in FIG. 13B or decrease the axial expansion/compression as illustrated in FIG. 13C.

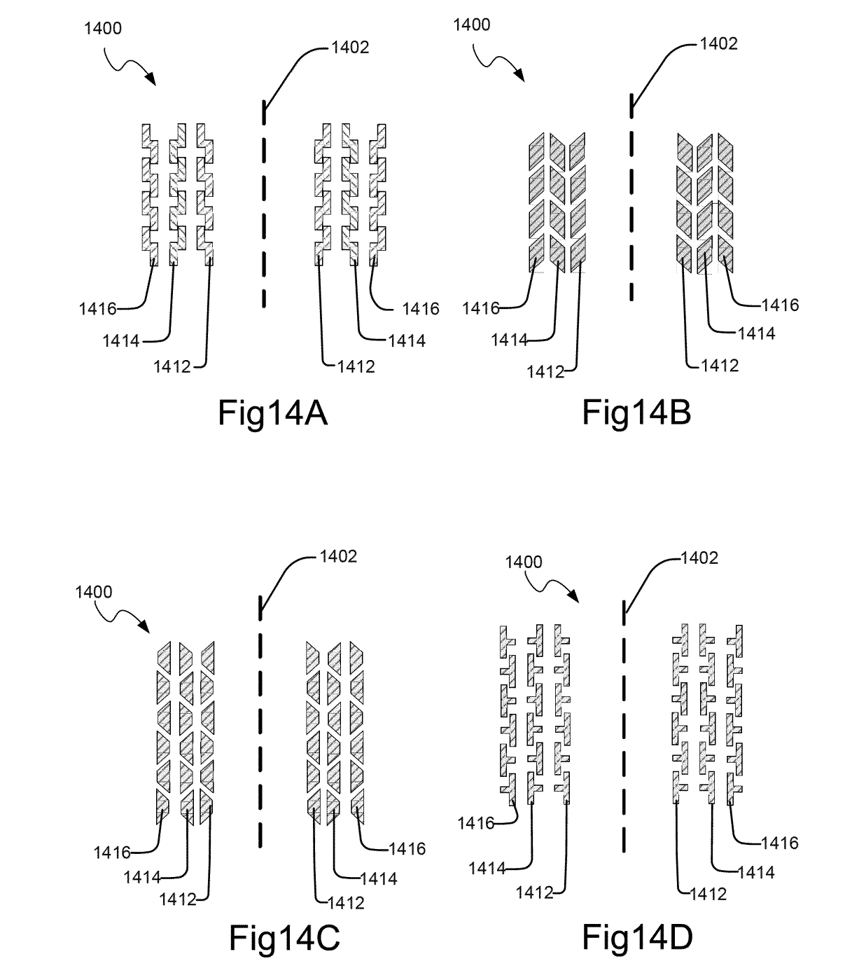

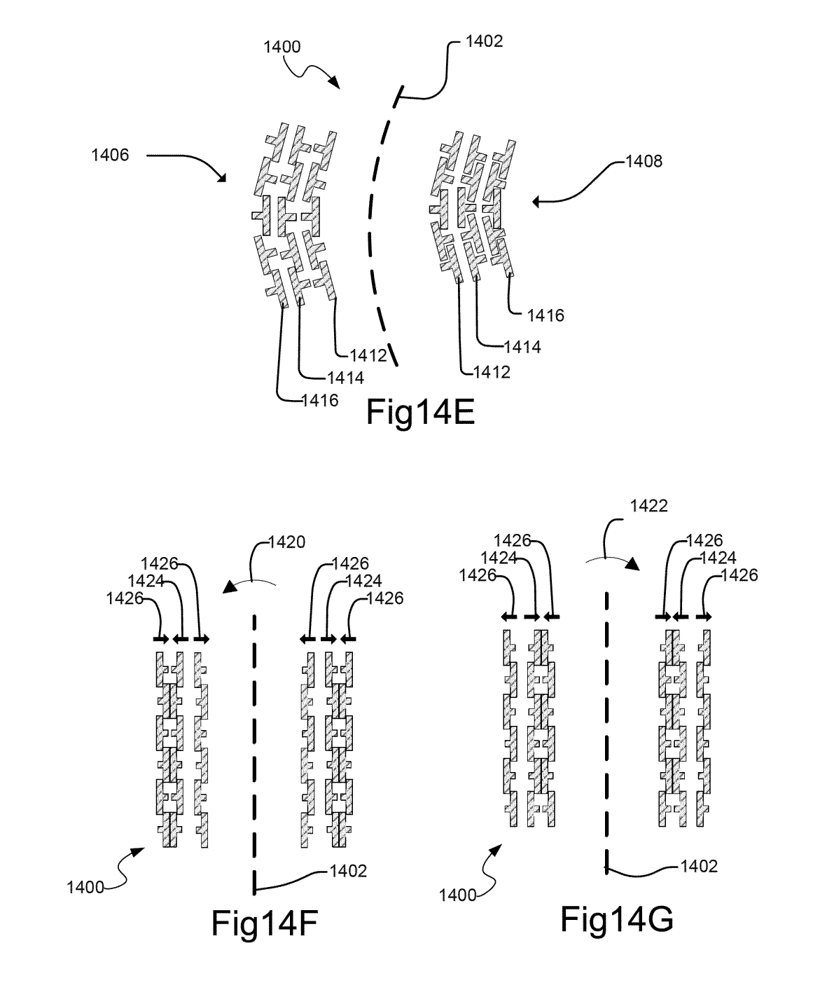

Referring back to FIGS. 4C and 5C, any of the coil constructions (i.e. single filar step shaped, single filar parallelogram shaped, double filar trapezoidal or double filar t-shaped) may be used in a bi-coil (400) or tri coil (500) configuration to allow for desired torsional stiffness or induced torsional stiffness. FIGS. 14A-14D illustrate cross sectional views of tri-core coils (1400) comprised of various cross sectional wire shaped filar coils. In each of these embodiments, the middle (1414) coil may be wound in the opposite direction as the inner (1412) and outer (1416) coils. The neutral axis (1402) represents the central axis of the tri-coil (1400). FIG. 14E illustrates the tri-coil (1400) in bending where the coils on the inner bend (1408) compress axially while the coils on the outer bend (1406) expand. Despite the compression or expansion, the axially adjacent windings in each coil of the tri-coil continue to overlap as shown. FIGS. 14F-14G illustrate the tri-coil (1400) when a rotational force is applied. Because each coil in the tri-coil is alternately wound, when the tri-coil is rotated in one direction, for example counter-clockwise (1420) as shown in FIG. 14F, the middle coil (1414) expands radially (1424) while the inner (1412) and outer (1416) coils contract (1426). Alternatively, FIG. 14G illustrates rotation in the clockwise direction (1422) where the middle coil (1414) contracts (1426) while the inner (1412) and outer coils (1416) expand (1424). In both cases illustrated in FIGS. 14F and 14G, two coils interfere preventing or opposing either coil from further expansion or contraction. The reactions to rotation of the tri-coil when in a straight position would be similar if the tri-coil was bent and then rotated. The coil construction is not limited to one, two, or three coils. In certain embodiments, any number of coils may be used to create varying degrees of radial and axial stiffness.

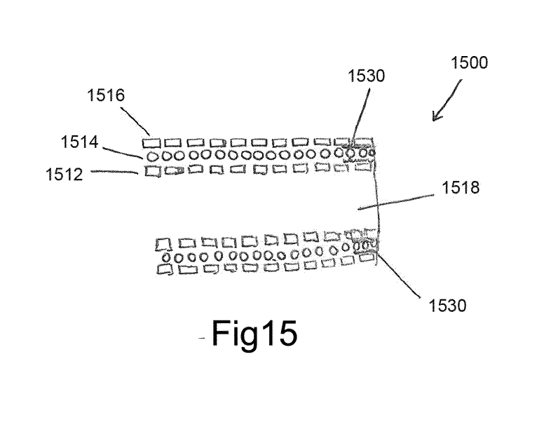

FIG. 15 illustrates another embodiment of a tri-coil structure of an elongate body (800). The tri-coil structure includes an outer coil (1516), a middle coil (1514), an inner coil (1512), and a lumen (1518). The outer coil (1516) may be constructed from a substantially flat wire which may provide substantial column strength as well as keeping or maintaining other internal coils contained or restrained during flexing, steering, bending, etc. The middle coil (1514) may be constructed from a substantially round wire to allow greater flexibility for flexing, steering, bending, etc. The inner coil (1512) may be also constructed from a substantially flat wire to further provide column strength support and to maintain or restrain the tri-coil structure by keeping the middle coil (1514) in place. The coil assembly may be welded at the end portions (1530) to maintain the coil assembly and to keep them in radial compression with one another. Different wire sizes, cross-sections, and coiling filars may be used to adjust or customize column strength, axial stiffness, and torsion stiffness of the coils. Furthermore, in a tri-coil structure, the coils may have alternating clockwise or counter-clockwise windings to provide torsional stiffness as the tri-coil structure is operated, steered, bent, flexed, etc.

The wires of the various coils may not be limited to shapes described in the previous embodiments. Indeed the wires may be manufactured or fabricated in various geometries in order to enhance or customize operational or performance characteristics of each or combination of coils. The size of each wire for different coils may vary in cross section or the size of one wire may vary along its length as it is wound to create a single coil. The wires of the coils may be manufactured from metal, polymers, ceramics, or any other suitable materials. The wires may be wound such that the spacing between each winding varies along the length of the coil. And any combination of the parameters including but not limited to wire cross-sectional shape, size, wire material, and spacing between windings or wires may be used to create a coil configuration that optimizes desired bending flexibility, axial compliance and torsional stiffness.

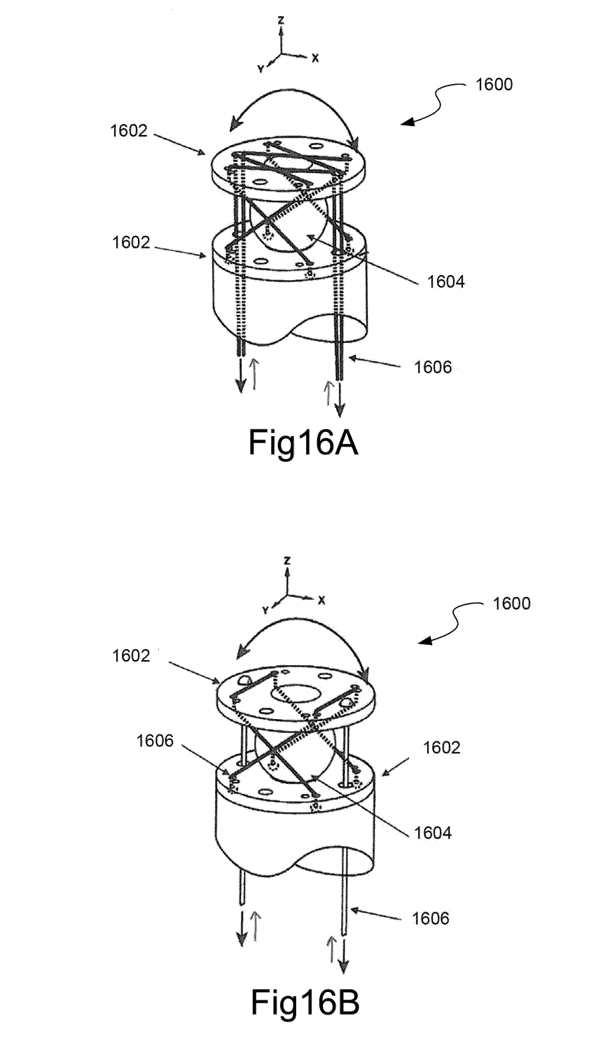

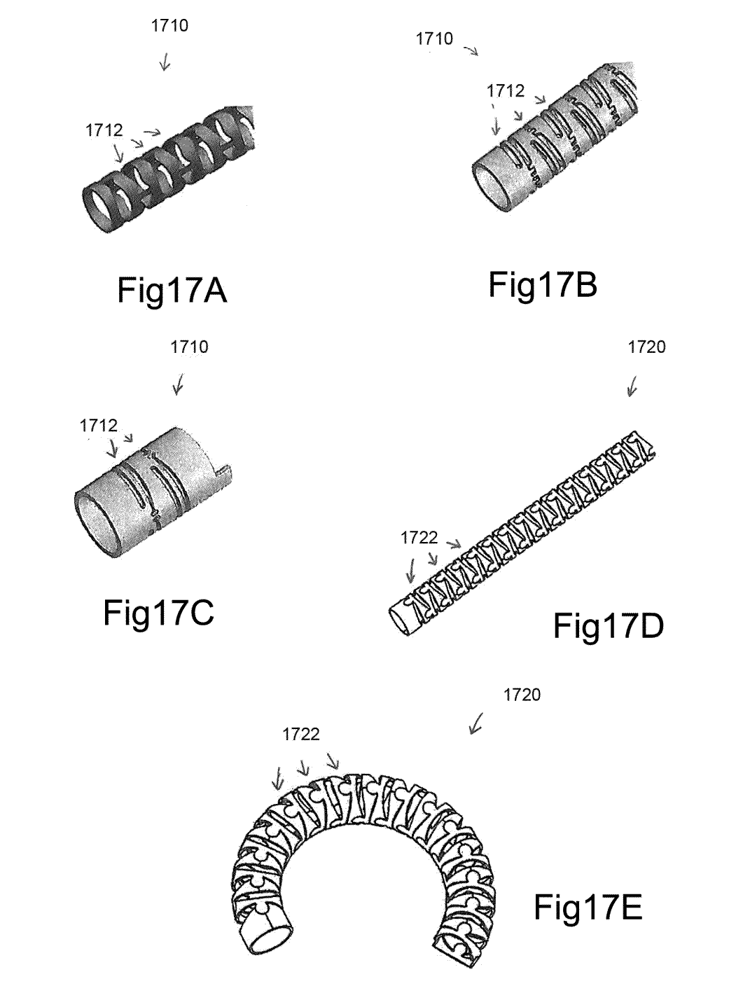

FIG. 16A through FIG. 17E illustrate various other embodiments of structural elements or members that may be configured with an elongate body to provide flexible steering and bending movements and rigid or stiff rotational or torsional support to transmit rotation or torque or torsion with no or substantially negligible or a reduced amount of torque or torsion lag or wind up. In this manner, movement or motion control input provided at the proximal portion of the elongate body may result in accurate and predictable movement or motion output at the distal portion of the elongate body. The ability to control the movement of a distal portion of an elongate body is particularly important when using the elongate body as an extension tool to reach tissue structures inside a patient for minimally invasive surgical procedures.

FIG. 16A and FIG. 16B illustrate a “ball-and-socket” structure (1600) which provides substantial flexible support for steering and bending movement and substantial rigid or stiff support against torsion for an elongate instrument. As illustrated in FIG. 16A and FIG. 16B, the ball-and-socket structure (1600) includes a plurality of platforms (1602) and at least one pivot element (1604) in which platforms may be pitched or pivoted in various directions. In one embodiment, the pivot element (1604) may be a ball-shaped element. The movements of the platforms may be controlled by operating one or more control elements (1606). As illustrated in FIG. 16A and FIG. 16B, the control elements (1606) may be coupled to the platforms in various configurations or patterns. The combination of platforms (1602), pivot element (1604), and control elements (1606) allows the ball-and-socket structure (1600) to be substantially flexible in various pitch and yaw movement and substantially stiff in torsional or rotational movements to prevent or minimize torque or torsion lag or wind up. As such, the ball-and-socket structure may be used as a structural element in an elongate body as an extension tool to control placement of surgical instruments or tools at a distal portion of an elongate instrument while providing steering or manipulation control input at a proximal portion of the elongate instrument. Alternatively, a plurality of ball and socket structures may be coupled in series to create a substantially flexible elongate member with both articulation means and torsional control. A hole in each platform and ball may provide a lumen for the elongate member. FIGS. 17A-17E illustrate other embodiments of support structures for elongate instruments in which the support structures may allow substantially flexible movements in steering, bending, or manipulation in various directions and be substantially stiff in torsion or rotation. In other words, all of these support structures may allow steering, bending, manipulation, or articulation of various portions of the support structure while also allowing tip or distal portion rotation or torsion indexing in accordance with control or manipulation of the base or proximal portion of the elongate instrument.

As illustrated in FIGS. 17A-17C, the support structures (1710) may be manufactured or fabricated from a tubular element. Various patterns of feature elements (1712) may be removed or cut-out of the tubular element to allow for flexible steering, bending, or articulation of the support structure while maintaining torsional or rotational stiffness. These support structures may be considered as “flexible spines”. Incorporating a flexible spine in a push-pull system may allow the support structure (1710) to articulate while also allowing the tip or distal portion of the support structure to be indexed to the base or proximal portion of support structure. In other words, the support structures in accordance with embodiments described herein allow precise control and placement of the distal portion of the elongate instrument by various control input at the proximal portion of the elongate instrument. The tubular elements may be manufactured or fabricated from various suitable materials. In some embodiments, the tubular elements may be manufactured or fabricated from a material with shape memory properties (e.g., nickel-titanium alloy) which may be used to form a flexible member with a particular pre-determined bias or shape such that the support structure (1710) will have a tendency to return to the pre-determined bias or shape.

Referring to FIG. 17D, the support structure (1720) may be comprised of coupled or interlocking segments (1722) to form a complete support structure. These segments (1722) may include features or patterns to allow coupling or interlocking of adjacent or adjoining segments. The features or patterns may allow movement or articulation between adjacent or adjoining segments such that only minimum amount of force may be necessary to steer or articulate various portions of the support structure (1720). In addition, the coupled or interlocking segment (1722) may also maintain axial and rotational integrity such that there may be no or negligible or a reduced amount of torque or torsion lag or wind up from the proximal portion of the support structure to the distal portion of the support structure. In this manner, movement or motion control input provided at the proximal portion of the support structure (1720) may result in accurate and predictable movement or motion output at the distal portion of the support structure (1720). FIG. 17E illustrates an example of an articulated support structure (1720) in which the interlocking segments (1722) maintain the integrity of the support structure while allowing articulation as well as rotational support.

In another embodiment, a mesh layer or braid layer may be added to any elongate flexible member, e.g., such as those illustrated in FIG. 16A through FIG. 17E or any conventional catheter or endoscope, to provide structural stability and further increase control, axial stiffness and flexibility, in particular in a push-pull system as the support structure is subjected to articulation or rotational loads. Referring back to FIGS. 17D and 17E, with an additional mesh or braid layer each individual strand of a mesh layer may act as a tether between an individual segment and its adjacent segments to resist or support rotational loads.

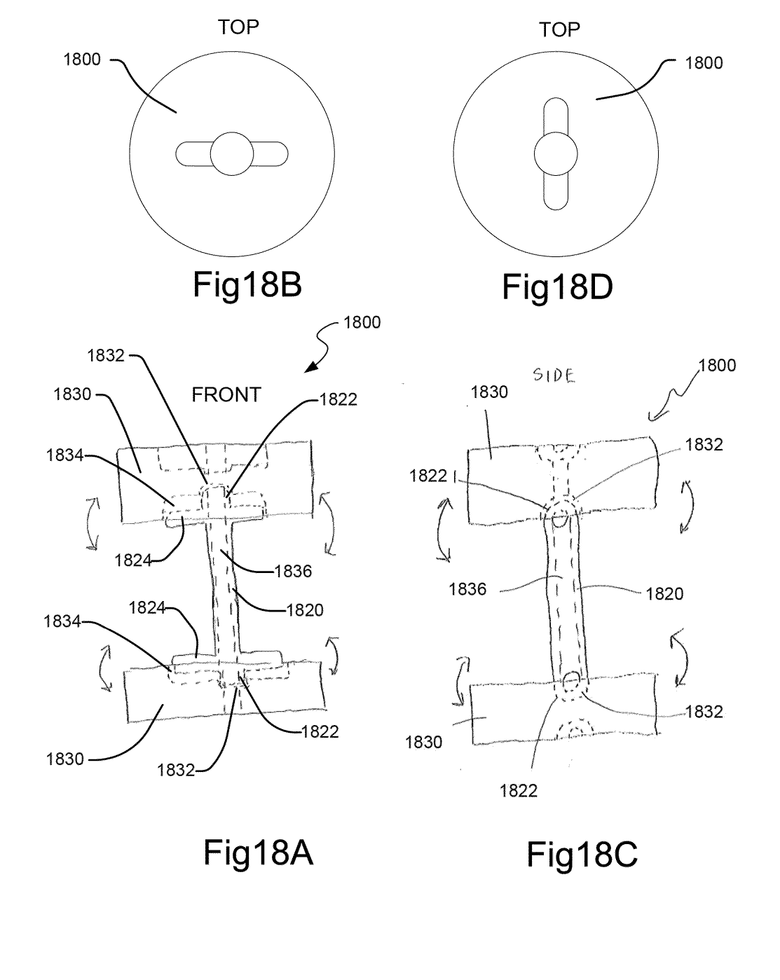

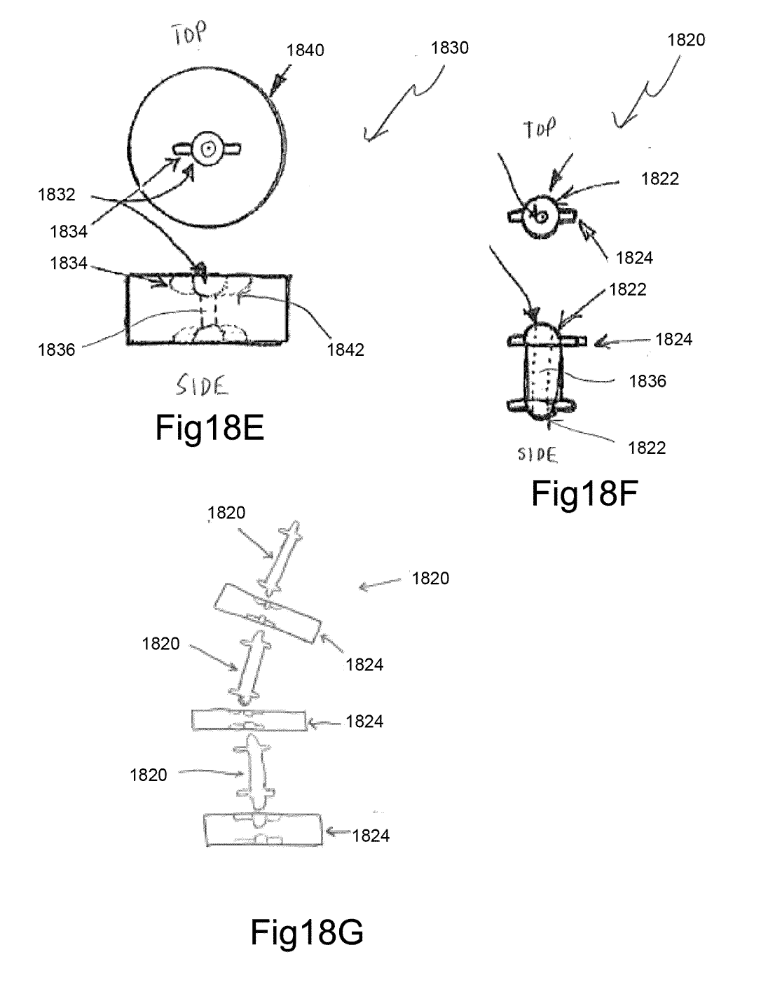

FIGS. 18A-12D illustrate a “spacer-segment” device (1800) that may be used to provide torsional stiffness when added to an elongate flexible member. FIG. 18A illustrates a front view, FIG. 18B illustrates the top view of FIG. 18A, FIG. 18C illustrates a side view, and FIG. 18D illustrates the top view of FIG. 18C.

FIGS. 18E and 18F show top and side views of the spacer (1830) and segment (1820) elements respectively. The spacer (1830) may be a disc (1840) with a spherical socket (1832) and torque or torsion transmission slot (1834) on its top and bottom sides. The torque or torsion transmission slot (1834) may be shaped such that from a top view, the slot may be substantially rectangular but the slot may be cut into the spacer with a hemispherical cut (1842) as shown in FIG. 18E. The segment (1820) may be a substantially rigid cylinder with substantially spherically shaped ends (1822) and torque transmission pins (1824).

Referring back to FIGS. 18A-18D, each segment (1820) may fit between two spacers (1830) with each of the spherical ends (1822) may rest in a spherical socket (1832) as the torque transmission pin (1824) fits in the torque transmission slot (1834). The segment ends and, the sockets may be spherically shaped, so the segment (1820) may pivot in the socket much like a ball joint and the torque transmission pin may rotate into the hemispherical slot allowing pivot in both the yaw and pitch directions. However, since the torque transmission sockets have a substantially rectangular shaped top profile, the segments (1820) may be prevented from twisting about its own longitudinal axis. Additionally thru-holes (1836) through each spacer (1830) and through each segment (1820) along its axis may provide a lumen for wires, cables, tools, etc.

By placing a plurality of spacer-segment devices (1800) in series, a substantially torsionally stiff support structure (1802) may be provided along the entire length or a portion of a length of an elongate device as shown in FIG. 18G. FIG. 18G illustrates an exploded view of the support structure (1802). The support structure (1802) may support various steering, bending, or articulation movements as well as provide torsional stiffness as described previously.

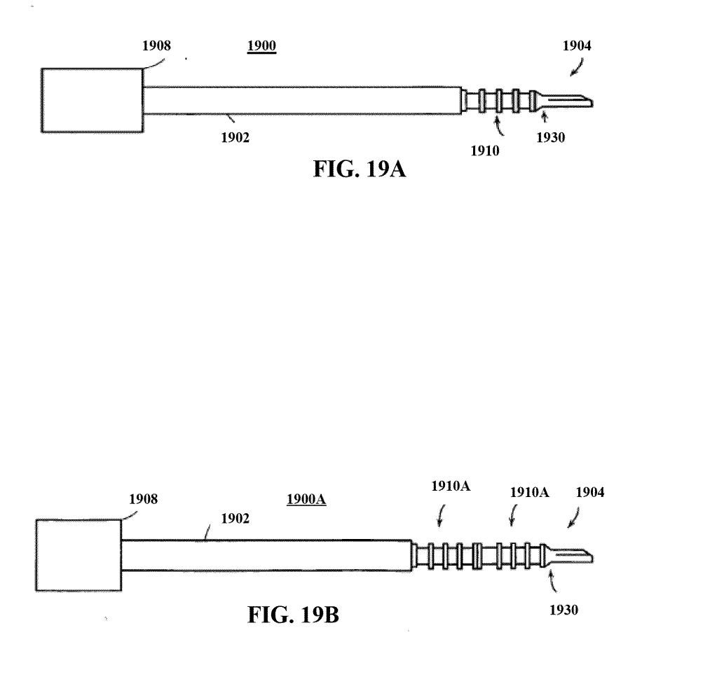

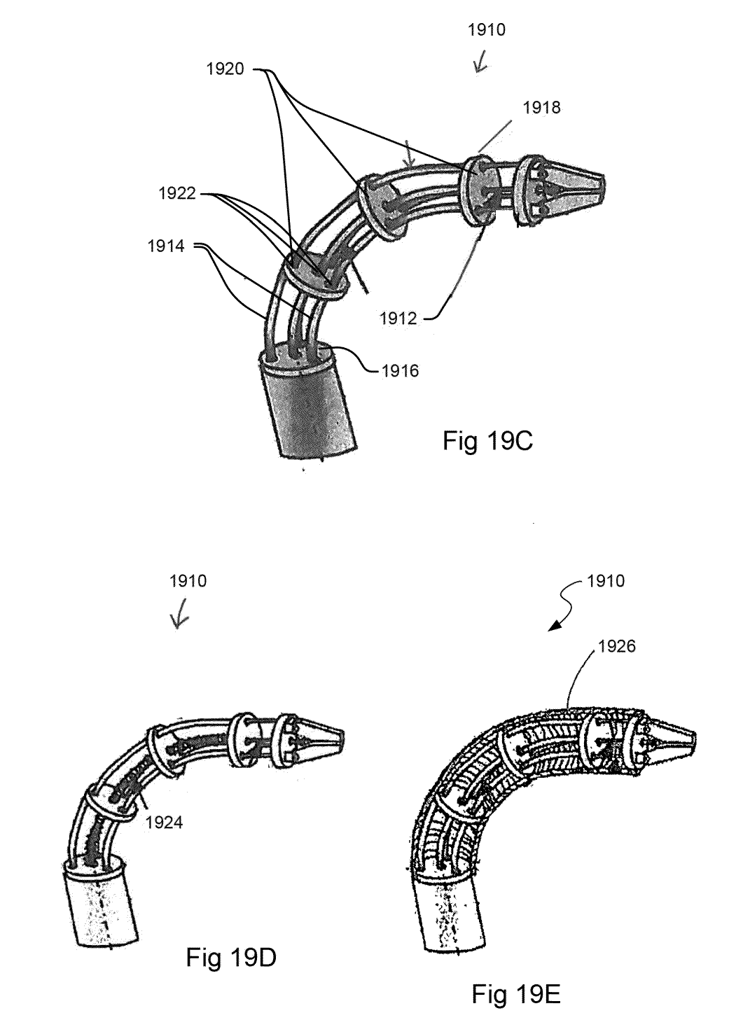

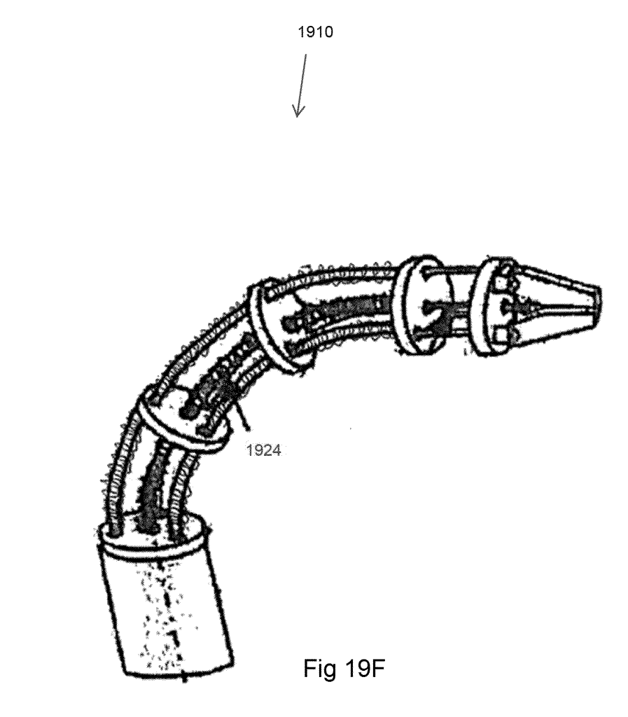

In addition to the aforementioned elongate instruments, the various elongate bodies (e.g., bi-coil structure, tri-coil structure, etc.) and support structures (e.g., tubular elements, segmented elements, etc.) may be incorporated or implemented into various other elongate instruments. For example, the elongate bodies or support structures may be incorporated or implemented into the elongate instrument shown in FIG. 19A.

As shown in FIGS. 19A-19B, in certain embodiments a distal dexterity device 1900 is provided which includes a holder member 1902, the operable end 1904, an actuation unit 1908, wires 1906 and wire ends 1907. The operable end 1904 also is configured and arranged so as to include a manipulation device 1910. A manipulation device may be, e.g., a flexible elongate body or flexible elongate instrument. The manipulation device 1910 shown in FIGS. 19A-19B is a manipulation device, similar to the manipulation device shown in FIG. 19C, which does not include or show the support members described herein. However, it is contemplated that the support members described herein, e.g., the support members shown in FIGS. 19D-19F, may be used to modify or combine with the manipulation device 1910 and the dexterity device 1900. In certain embodiments, a tip member or wrist device 1930 may also be included. The distal dexterity devices provide the necessary flexibility for bypassing obstacles as the operable end 1904 is traversing the pathway to the surgical site.

The manipulation device 1910 and the actuation unit 1908 are operably coupled to respective ends of the holder member 1902. In particular embodiments, the holder member 1902 is a tubular member (e.g., thin tube) of a biocompatible material characterized as having sufficient strength to withstand the loads imposed during a procedure/technique as the holder Member is being rotated or moved axial by the manipulation unit 1906 and when the actuation device 1908 is acting on the manipulation device 1910 for re-configuring (e.g., bending) the manipulation device. The lumen within the holder member 1902 also establishes a pathway through which the secondary back bones or axially extending members 1914 along with any internal wires 1906 run between the manipulating device 1910 and the actuation device 1908. This preferably also creates a barrier between the axially moving elements of the distal dexterity device and the surrounding tissues. The width and length of the holder member 1902 may be set based on the particulars of the procedure to be performed. For example, in throat surgical procedures it is desirous for the operable end 1904 of the distal dexterity device to extend about 180-250 mm into the throat. Thus, the length of the holder member 1902 would be set so as to accomplish this. Similarly, the width of the holder member 1902 is set based on the size of the opening, the size of the passage, the area available at the surgical site and the interior dimensions of the member that is typically inserted into the opening (e.g., laryngoscope).

The holder member 1902 may be in the form of a rigid tubular element, a flexible tubular element or device or is composed of rigid tubular portions and flexible tubular portions to fit the use and function of a distal dexterity apparatus. For example, the portion of the holder member 1902 that is disposed with a device manipulation unit may be a rigid member and other portions of the holder member may be flexible in construction. The flexible portions of the holder member 1902 can comprise for example, a flexible device such as a catheter, flexible endoscope, or another snake-like unit. In another example, the external portion of the holder member 1902 would comprise a flexible portion and the remainder of the holder portion including the portion within the patient would comprises a rigid portion.

Also, when the secondary backbones or axially extending members 1914 pass through a flexible portion of the holder member 1902, the secondary backbones may be constructed or selected from materials and structures that are flexible in bending for those portions that remain inside the holder member 1902 but still stiff in the axial direction for transmission of force in either a push or pull direction. In further embodiments, the secondary backbones or axially extending members 1914 are supported in flexible sheaths so as to further prevent buckling in a long flexible section. Such a structure advantageously yields a system that is useable in flexible endoscopy applications and also in intracavitary procedures such as ablations inside the heart. Such a design also advantageously allows multiple snake-like units to be placed sequentially in order to further increase dexterity, as discussed further herein.

As shown in FIG. 19C, in certain embodiments, the manipulation device, e.g., a flexible elongate body, 1910 includes a base disk or member 1916, an end disk or member 1918, intermediate spacer disks or members 1920, a central backbone 1912, and secondary backbones 1914. The foregoing are arranged and configured so as to yield a snake-like unit that is generally categorizable as a continuous non-extensible multi-backbone unit.

The central and secondary backbones or axially extending members 1912, 1914 are generally in the form of a flexible tubular member, such as a super-elastic tube, more specifically a tube made from NiTi. More generally, the secondary backbones or axially extending members 1914, are constructed or selected from materials and structures (e.g., diameter and wall thickness) so that they are flexible in bending but still stiff in the axial direction for transmission of force in either a push or pull direction and the central backbone are constructed or selected from materials and structures (e.g., diameter and wall thickness) so it flexible in bending. Also, such materials and structures preferably yield a member that does not permanently deform (e.g., buckle) when the manipulation device 1910 is being manipulated or bent.

In certain embodiments, there are three secondary backbones or axially extending members 1914 arranged about and spaced from the central backbone or axially extending member. It should be recognized that the number of secondary backbones or axially extending members 1914 is set so as to provide the required bending motion while generally assuring that the central and secondary backbones or axially extending members 1912, 1914 do not permanently deform (e.g., buckle) when the manipulation device 1910 is being manipulated or bent. The central tube or backbone or axially extending member 1912 is the primary backbone or axially extending member while the remaining three tubes are the secondary backbones or axially extending members 1914. In illustrative exemplary embodiments, the secondary backbones or axially extending members 1914 are spaced equidistant from the central backbone or axially extending member 1912 and from one another.

The central backbone or axially extending member 1912 may be attached or secured to the base member 1916 and the end member 1918 as well as to all of the intermediate spacer members 1920. The secondary backbones or axially extending members 1914 may be attached to the end member 1918 and are slidably disposed within apertures 1922 provided in each of the base member 1916 and the intermediate spacer members 1920. In this way, the secondary backbones or axially extending members 1914 are free to slide and bend through the apertures 1922 in the base member 1916 and intermediate spacer members 1920. As herein described, the secondary backbones or axially extending members 1914 are used for actuating the manipulating device 1910 using one or a combination of both push and pull modes and also pass through the lumen or guiding channel(s) in the holder member 1902.

The intermediate spacer members 1920 are configured and arranged (e.g., spaced from one another) to prevent buckling of the central and secondary backbones or axially extending members 1912, 1914 and to maintain an equal distance between the secondary backbones or axially extending members and the central backbone or axially extending member. In further embodiments, the intermediate spacer members 1920 are placed close enough to each other so that the shapes of the primary and secondary backbones or axially extending members 1912, 1914 are constrained to lie in a prescribed fixed distance apart. The intermediate spacer members are also arranged and fixed on the central backbone or axially extending member 1912 such that they do not prevent the central backbone or axially extending member from bending while providing negligible friction to movement of the secondary backbones or axially extending members 1914.

In certain embodiments, the secondary backbones or axially extending members 1914 are sized so as to have the same size as the primary backbone or axially extending member and therefore their bending properties are significant (i.e. they can not be treated as wires). This allows the manipulation device 1910 to be constructed so as to have a small diameter for use in confined spaces such as the throat while maintaining structural rigidity and simplicity of actuation. Also, by using push-pull elements for the actuation of the manipulation device 1910, it is possible to satisfy the statics of the structure while preventing buckling of the backbones or axially extending members. This also allows the diameter of the manipulation device 1910 to be reduced such as for medical applications requiring a diameter smaller than 4 mm.

The above described structures yield a snake-like device that embodies a flexible backbone system made up of a plurality or more of backbones or axially extending members 1912, 1914 that can advantageously achieve high structural stiffness in bending and torsion, particularly when compared to that achievable with conventional systems that embody wires. Further, such a flexible backbone system advantageously eliminates small precision joints as required with conventional systems that embody articulated joints, thereby reducing manufacturing costs and avoiding designs issues associated with backlash.

As herein described, the backbones or axially extending members 1912, 1914 also are configured and arranged so as to have more that one usage or function other than the above-described structural use (i.e., dual usage). The lumen or internal passage of the backbones or axially extending members 1912, 1914 are adaptable so as to be used to provide a pathway for passage of a fiber optic cable for example that can be used as a light source to illuminate the treatment site for visualization of the treatment site and the operable ends 1904 of the distal dexterity devices 1900.

The lumen or internal passage of one or more backbones or axially extending members are useable as a fluid passage for delivering fluids such as for delivery of a therapeutic medium or aspiration as well as for suctioning away fluid and/or debris. In such a case, it is contemplated that a given backbone or axially extending member 1912, 1914 and the distal dexterity device 1900 would be adapted so as to be capable of performing these function(s). For example, the distal dexterity device 1900 would be adapted so that the backbone or axially extending member internal passage would be fluidly coupled to an external source of fluid and/or suction source and the operable end 1904 thereof would be adapted for delivery of the fluid and/or suction. Also for example, one secondary backbone or axially extending member 1914 could be configured to fluid delivery while another backbone or axially extending member, such as the centrally located backbone or axially extending members 1912, could be fluidly coupled to a suction source.

Additionally, the lumen or passage way of one or more backbones or axially extending members 1912, 1914 are useable as a passageway in which passes the actuating members (i.e., wires 1906) that comprise a mechanism to operably couple the wrist unit 1930 and the manipulating device 1910. The lumen or internal passages of one or more backbones or axially extending members 1912, 1914 of a first manipulating device 1910a also are useable as a passageway for the secondary backbones or axially extending members 1914 of a second manipulating device 1910b.

In further embodiments, and with particular reference to FIG. 19B, the distal dexterity device 1900a is configured and arranged so as to include two or more manipulating devices 1910a, b that are stacked one upon the other. In such an application, the secondary backbones or axially extending members 1914 for the second manipulation device 1910b or second section would be passed through the secondary backbones or axially extending members of the first manipulation device 1910a or first section. This allows for serial stacking of the second section on the first section and also creates a multi-section snake that can be used for exploration and surgical intervention in deeper regions such as through the airways of the lung. Such stacking also necessarily allows each of the manipulation devices 1910a, b to be actuated independent of each other. Thereby allowing the distal dexterity device to achieve or be capable of exhibiting two additional degrees of freedom for each manipulation device provided, while at the same time not being subjected to the limitations or concerns that are created with the addition of articulate joints in conventional systems.

In sum, an advantageous effect that flows from the architecture of the manipulation device 1910 stems from the use of flexible backbones, thereby removing the dependency on small universal joints and wires as with conventional devices and systems. In addition to reducing manufacturing costs of the manipulation device as compared to conventional devices and this contributes to the possible reduction in size due to the small number of moving parts and the absence of standard miniature joints. Another advantage effect comes from the secondary use of tubes for the backbones or axially extending members, thus providing a secondary application for these backbones or axially extending members. As indicated herein these backbones can serve as suction channels, fluid channels, an actuation channel for the tool mounted on its distal end or as a source of light for imaging. In a particular embodiment, a mechanism (e.g., wire, tube) is passed through the central backbone or axially extending member 1912 which mechanism is used to actuate or control the operation of the surgical tool/instrument/device associated with a given distal dexterity device 1900.

In another embodiment, the manipulation device 1910 is configured and arranged so that one of the secondary backbones or axially extending members 314 is a redundant secondary backbone or axially extending member, which can be actuated to reduce the amount of force acting on the primary backbone or axially extending member and by doing so, reducing the risk of its buckling.

Referring again to FIG. 19C, which shows a construction for a flexible elongate instrument or elongate body or manipulation device (1910) which provides bending and steerability with the use of tubular backbones. The instrument (1910) includes a base disk (1916), an end disk (1918), intermediate spacer disks (1920), a central backbone (1912), and secondary backbones (1914).

The secondary backbones (1914) should be constructed from a material which provides for flexibility in bending but stiffness in the axial direction allowing for transmission of force in either the push or pull direction. The central backbone (1912) should be constructed of a material which provides for flexibility in bending. In one embodiment the central and secondary backbones may be constructed of the same material such as NiTi but formed in different constructions (i.e. varied diameter or wall construction) to provide for bending flexibility for all backbones but axial stiffness for only the secondary backbones. Though only two or three secondary backbones are shown in FIG. 19C, a plurality of secondary backbones may be used to achieve the desired bending motion while preventing buckling of the elongate instrument. In exemplary embodiments, there are three secondary backbones arranged equally spaced about the central backbone.

The central backbone (1912) may be secured to the base disk (1916), end disk (1918) and all intermediate disks (1920). The secondary backbones (1914) may be secured only to the end disk (1918). The intermediate and base disks (1920, 1916) are constructed with thru-holes (1922) such that the secondary backbones (1914) are slideably disposed within the holes (1922) and the secondary backbones (1914) are free to slide and bend through the intermediate and base disks (1920, 1916). The intermediate disks (1920) are positioned axially spaced to prevent buckling of the central and secondary backbones (1912, 1914) while also maintaining equal distances between the secondary backbones and the central backbone (1912,1914).

The above described constructions provide for a snake-like, steerable flexible elongate members, bodies, instruments or manipulation device, which may be used in combination with the dexterity devices described above. The secondary backbones (1914) provide push pull members which can be used to steer the distal tip of the flexible elongate member or body or manipulation device (1910) and the central backbone (1912) provides structural stiffness in bending. While the central backbone (1912) can also provide for some stiffness in torsion depending on the construction of the central backbone and the length of the elongate body or member, it is still a thin tube like structure which functionally provides decreasing stiffness in torsion as the length of the elongate body or member increases. Thus additional support structures or members as described herein should be added or incorporated into the elongate member or elongate body or manipulation devices (1910) and the dexterity devices (1900) described above, which do not include or show such support members, in order to modify such elongate bodies or devices to provide adequate torsional stiffness and stability thereto.

As illustrated in FIG. 19D-19F, support structures may be incorporated or implemented in various elongate instruments or elongate bodies or manipulation devices (1910) to support various steering, bending, or articulation movements while also resisting or transmitting torsion or rotation. In other words, the support structures or members will allow movement or motion control input provided at the proximal portion of the flexible elongate instrument or body (1910) to cause accurate and predictable movement or motion output at the distal portion of the elongate instruments or bodies (1910). The support members provide torsional stability and transmit torsion from a proximal end to a distal end of an elongate body or device with no, negligible, reduced or minimal torsion lag or wind-up while maintaining flexibility of the elongate body.

FIG. 19D shows the use of a helical member, e.g., a tri-coil support structure (1924), surrounding the central backbone (1912) of the flexible elongate device (1910) of FIG. 19C which provides for torsional support. FIG. 19E shows the flexible elongate device (1910) surrounded with a mesh or braided cover (1926) to provide torsional support. In another embodiment, a mesh or braided cover may surround one or more of the individual axially extending members. FIG. 19F shows the use of helical members, e.g., a bi- or tri-coil, as support members where a separate helical support member surrounds the central backbone and each of the secondary backbones or axially extending members.

Other embodiments could include but are not limited to the use of flexible spines, support structures, ball and socket elements, and spacer-segment devices to provide torsional support to the flexible elongate device or body (1910), and thus, provide torsional support to the dexterity devices 1900 which may incorporate such flexible bodies, members, or devices. In certain embodiments, any combination of the previously described mechanisms for torsional stiffness or induced torsional stiffness may be used to increase the desired torsional stiffness of the elongate instrument and do not need to be limited along the length of the instrument. As a non-limiting example, a ball and socket platform may be used at the distal tip of the mechanism, while a tri-coil may be placed at a portion of the distal section while the remaining length of the instrument may include a braided layer. Thus, any combination of mesh, braid, support structure, ball and socket platform or coil configuration as previously described may be used to increase the torsional stiffness of any elongated flexible member or body including but not limited to catheter, endoscopes, cables, wires, tubing, etc. One, two or up to any combination of apparatus may be implemented either along the entire length of an elongate member or body or on any distinct portion of the elongate member or body depending on desired torsional and axial stiffnesses as may be required for a particular application. Varying the type of torsionally stiff device in combinations along the length of an elongate member or elongate body may vary both the torsional stiffness and the bending flexibility and compression of an elongate device along its body length.

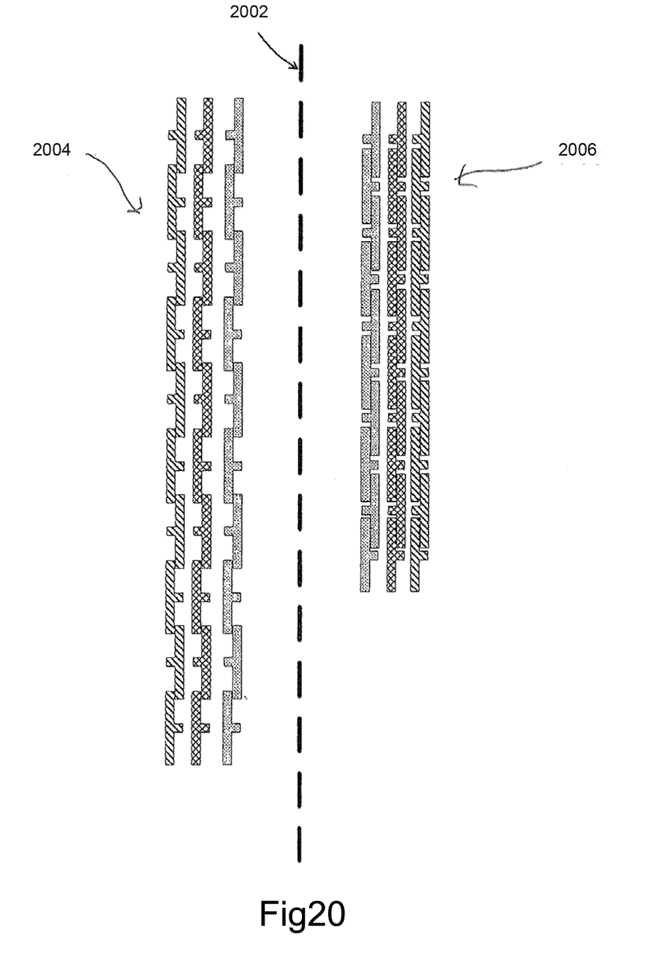

As illustrated in FIG. 20, as an elongate body is steered, bent, or navigated, the windings of the coils on one side of the neutral axis (2002) may be slightly compressed (2006) while the windings of the coils on the other side of the neutral axis might slightly expand (2004). In one embodiment, steering of the elongate member may be accomplished using a plurality of pull wires connected to the distal tip of the elongate member that run the length of the elongate member through lumens in the elongate member walls. As previously described for catheter control, as a pull wire is pulled in tension, the distal tip of the elongate member such as a catheter may bend in the direction of the pull wire. In order to obtain steering control of the catheter, a method of control may be to tension all the pull wires such that the catheter is axially compressed, and then release the pull wire on the side opposite the desired direction of bending. Thus axial compressibility is desirable for a pull wire steering modality in articulating sections of the elongate member. Because of the necessity to control steering articulation at the distal tip, a portion of the length of the elongate member may be considered as an articulation section and would require a different axial stiffness than a non-articulating section of the elongate member. Using any combination of the previously disclosed torsional stiffness apparatus, an ideal balance between torsional stiffness, axial stiffness, and bending flexibility may be obtained at necessary locations along the length of the elongate member.

In an alternate steering embodiment, push tubes may be used in addition to pull wires for steering. In this embodiment, axial expandability may be desirable for articulating sections of the elongate member.

In certain embodiments, including any of the embodiments described herein, various portions of an elongate flexible member, such as catheter, elongate body or elongate instrument may take on a variety of shapes, sizes, and/or dimensions to provide for varying degrees of movement of the support member, catheter, elongate body, elongate instrument, or other devices incorporating the coils. In certain embodiments, the diameter of a coil wire may range from about 0.001 to about 0.01 inches. In certain embodiments, the spacing between axially adjacent windings of a coil may range from about zero to about 0.001 inches. In certain embodiments, the spacing between radially adjacent windings of radial adjacent coils or the radial spacing between radially adjacent coils may range from about 0.001 to about 0.01 inches. In certain embodiments, the total thickness of a wall of an elongate flexible member, e.g., a catheter, elongate body, or elongate instrument, may range from about 0.001 to about 0.01 inches. In certain embodiments, the bend radius of an elongate flexible member, e.g., a catheter, elongate body, or elongate instrument, may range from about 1 mm to about 25 mm.

The dimension of various portions or sections of an elongate flexible member, a catheter, support member, elongate body, or elongate instrument may vary. For example, in certain embodiments, the articulation section or distal section of a catheter or other elongate flexible member or support member may include the following dimensions: a coil wire diameter of about 0.002 inches; about 0.00025 inches of spacing between axially adjacent windings of a coil; about 0.001 inches of radial spacing between radially adjacent coils or spacing between radially adjacent windings of radial adjacent coils; a total wall thickness of about 0.008 inches; and/or a bend radius of about 10 mm. In certain embodiments, a non-articulation section of a catheter or other elongate flexible member or support member, which may be relatively stiff, may include a coil where the spacing between axially adjacent windings of the coil is near zero or as close to zero as possible to provide increased stiffness to the coil. Thus, the spacing between axially adjacent windings of a coil may vary along the length of any coil in an elongate flexible member, a catheter, support member, elongate body, or elongate instrument, (having tri- or bi-coils, or any number of coils) depending on the desired degree of bend and flexibility of a particular section of the elongate device.

In accordance with another embodiment, a method for performing minimally invasive surgical procedure includes inserting an elongate instrument through an incision or opening of an entry site. The elongate instrument includes a support member that allows at least one degree of freedom of movement of various portions of the elongate instrument. The method further includes advancing the elongate instrument along a pathway through the entry site, steering and guiding a distal portion of the elongate instrument toward a target tissue structure through the pathway, and operating an instrument that is operatively coupled to the distal portion of the elongate instrument to diagnose or treat the target tissue structure.

In certain embodiments, a method of performing a minimally invasive surgical procedure is provided. The method includes inserting an elongate instrument into a patient where the elongate instrument has an elongate body and a support member disposed within the elongate body. The support member may have a plurality of coils wherein at least two of the coils are wound in opposite directions and wherein a winding of at least one coil has features that overlay or interlay with axially adjacent windings of that coil. The elongate instrument is advanced along a pathway in the patient and a distal portion of the elongate instrument is steered and guided toward a target tissue structure through the pathway. An instrument that is operatively coupled to the distal portion of the elongate instrument may be operated to diagnose or treat the target tissue structure where torsion may be transmitted from a proximal end to a distal end of the elongate instrument or elongate body of the instrument with no or negligible torsion lag or wind-up.

In certain embodiments, a flexible elongate body is provided which includes one or more or a plurality of axially extending members and one or more support members wherein the support members are configured to provide torsional stability to the flexible elongate body. The flexible elongate body may also include a base member, an end member and one or more intermediate spacer members. In certain embodiments, at least one of the plurality of axial extending members may be secured to each of the base member, the end member and/or at least one of the intermediate spacer members. The other of the plurality of axial extending members may be secured to the end member and slidably disposed through apertures in at least one of the intermediate spacer members and the base member. The support members may allow torsion to be transmitted with no or negligible torsion lag or wind-up from a proximal end to a distal end of the elongate body. In certain embodiments, a support member is positioned along a length of at lest one of the axially extending members. Optionally, the support member is configured to surround or encapsulate at least one of the axially extending members or to surround or encapsulate the plurality of axially extending members. In certain embodiments, a support member may serve as an axially extending member.

In certain embodiments, a support member may include a plurality of coaxially arranged helical members including first and second helical members wound in opposing directions. The first helical member may have a first winding with features that overlay or interlay with features of an axially adjacent winding of the first helical member. The first and second helical members may be configured such that when a rotational force is applied to the flexible elongate body the first and second helical members are driven in opposing radial directions interfering with one another in opposing radial directions. The overlaying or interlaying of the axially adjacent windings of any of the helical members, e.g., the first helical member, will help to minimize or eliminate overlap or herniation between radially adjacent windings of the first, second or other helical members.

Optionally, one or more helical members may be wound from a wire having a cross sectional shape configured to provide overlapping or interlocking between axially adjacent windings of the respective helical member. The cross sectional shape of the wire may include but is not limited to a step shape, parallelogram shape, trapezoidal shape, or T-shape. The distance of spacing between axially adjacent windings of a helical member may vary along a length of the helical member such that bending of the flexible elongate body can be maximized or minimized along different portions of the body or device. Spacing between axially adjacent windings of a helical member may vary. For example, the spacing may have a distance ranging from about 0.00010 to 0.00045 inches. The bend radius of a flexible elongate body may vary. For example, the bed radius may range from about 7 mm to about 12 mm or more or less.

In certain embodiments, the axially extending members may be arranged so one or more of the axially extending members are disposed about and parallel to a centrally located axially extending member. For example, three secondary axially extending members can be disposed about and parallel to the centrally located axially extending member. The axially extending members may be configured and arranged so as to be flexible in bending and stiff in the axial direction so that the axially extending members do not deform when the elongate body is being manipulated. Optionally, an axially extending member may include a lumen configured to receive various tools or devices, such as an actuating member. The axially extending members may be configured and arranged so as to form a continuous extensible or non-extensible flexible backbone system capable of at least two degrees of freedom.

In certain embodiments, a tool may be operably coupled to a first end of the flexible elongate body and/or an actuation device may be operably coupled to a second end of the flexible manipulation device. The actuation device may be configured and arranged to cause the flexible elongate body to maneuver the operably coupled tool in one or more directions responsive to outputs of the actuation device.

In certain embodiments, a method of performing a minimally invasive diagnostic, surgical or therapeutic techniques is provided. The method may include inserting a flexible elongate body into a patient's body. The flexible elongate body may include one or more or a plurality of axially extending members and one or more support members. The support members may be configured to provide torsional stability to the flexible elongate body. The method may also include steering the elongate body from a first position to a second position in the body; transmitting torsion from a proximal end to a distal end of the elongate body with no or negligible torsion lag or wind-up while maintaining flexibility of the elongate body, e.g., by maintaining sufficient spacing between axially adjacent windings of a helical member in embodiments utilizing a helical member as a support member; and operating an instrument that is operatively coupled to a distal portion of the elongate body to diagnose or treat a target tissue structure in the body. In certain embodiments, the support member is positioned along a length of at lest one of the axially extending members. Optionally, the support member is configured to surround at least one of the axially extending members or to surround the plurality of axially extending members. In certain embodiments, a support member may serve as an axially extending member.

In certain embodiments, a support member utilized in the flexible elongate body may include one or more helical members; e.g., a first helical member positioned along a length of an axially extending member and a second helical member positioned along the length of an axially extending member. The method may also include actively driving the first helical member in a first direction; actively driving the second helical member in a second direction opposite the first direction such that the first and second helical members interfere with one another in opposing radial directions to provide torsional stability to the elongate body. Optionally, overlay or interlay may be allowed between features of axially adjacent windings of a helical member or between the windings themselves to prevent or minimize overlap between radially adjacent windings of the first and second helical members or other helical members.

Optionally, one or more helical members may be wound from a wire having a cross sectional shape configured to provide overlapping or interlocking between axially adjacent windings of the respective helical member. The cross sectional shape of the wire may include but is not limited to a step shape, parallelogram shape, trapezoidal shape, or T-shape. The distance of spacing between axially adjacent windings of a helical member may vary along a length of the helical member such that bending of the flexible elongate body can be maximized or minimized along different portions of the body or device. Spacing between axially adjacent windings of a helical member may vary. For example, the spacing may have a distance ranging from about 0.00010 to 0.00045 inches. The bend radius of a flexible elongate body may vary. For example, the bed radius may range from about 7 mm to about 12 mm or more or less.

In certain embodiments, the axially extending members may be arranged so one or more of the axially extending members are disposed about and parallel to a centrally located axially extending member. For example, three secondary axially extending members can be disposed about and parallel to the centrally located axially extending member. The axially extending members may be configured and arranged so as to be flexible in bending and/or stiff in the axial direction so that the axially extending members do not deform when the elongate body is being manipulated. The axially extending members and the base, end and intermediate spacer members may connected or arranged in various configurations as described above. Optionally, an axially extending member may include a lumen configured to receive various tools or devices, such as an actuating member. The axially extending members may be configured and arranged so as to form a continuous extensible or non-extensible flexible backbone system capable of at least two degrees of freedom.

In certain embodiments, a tool may be operably coupled to a first end of the flexible elongate body and/or an actuation device may be operably coupled to a second end of the flexible manipulation device. The actuation device may be configured and arranged to cause the flexible elongate body to maneuver the operably coupled tool in one or more directions responsive to outputs of the actuation device.

In certain embodiments, a steerable elongate instrument is provided which may include an elongate body and a support member disposed within the elongate body, the support member may include a plurality of coils wherein the plurality of coils comprise first and second coils wound in opposing directions. At least one coil may have a first winding with features that overlay or interlay with features of an axially adjacent winding of that coil or the windings themselves may overlay or interlay with one another. The first and second coils may be configured such that when a rotational force is applied to the elongate body the first and second coils are driven in opposing radial directions and interfere with one another in opposing radial directions. The overlaying or interlaying of the axially adjacent windings of a coil minimizes or prevents overlap between radially adjacent windings of the first, second or other coils to provide torsional stability to the elongate body.

In certain embodiments, the coils may be coaxially arranged. The coils may be configured such that torsion is transmitted with no or negligible torsion lag or wind-up from a proximal end to a distal end of the elongate body. Coils may be wound from a wire having a cross sectional shape configured to provide overlapping or interlocking between axially adjacent windings of a coil. The cross sectional shape of a wire may include various shapes, such as a step shape, parallelogram shape, trapezoidal shape, and T-shape. A coil may be wound from a wire having a cross sectional shape configured to provide overlap between axially adjacent windings of that coil where the overlap obstructs spacing between the axially adjacent windings such that overlap between radially adjacent coils is negligible or eliminated.

The distance between axially adjacent windings of a coil may vary along a length of the coil such that bending of the elongate instrument or body can be maximized or minimized in different portions of the elongate instrument or body. In certain embodiments a tri-coil configuration is provided. The tri-coil may be configured to provide torsional stability to the elongate body, e.g., by transmitting torsion with no or negligible torsion lag or wind-up from a proximal end to a distal end of the elongate body in at least two rotational directions. Optionally, the axially adjacent windings of a coil may link together to form a solid tube that allows for axial compression and expansion of the elongate body. Optionally, a support member may include coupled or interlocking segments.

In certain embodiments, a steerable elongate instrument is provided which has an inner, middle, and outer coil, wherein the middle coil is wound in the opposite direction as the inner and outer coils such that when a rotational force is applied to the elongate body the middle coil interferes with the inner or outer coils and opposes radial expansion and/or contraction of the inner or outer coil. Optionally, the inner and outer coils may be constructed from a substantially flat wire and the middle coil may be constructed from a round wire. A lumen may be provided within the inner coil. Spacing between axially adjacent windings of a coil may vary, e.g., it may range from about 0.00010 to 0.00045 inches and the elongate body has a bend radius that varies, e.g., the bend radius may range from about 7 mm to 12 mm.

In certain embodiments, a method of performing a minimally invasive surgical procedure is provided. The method may include: inserting an elongate instrument into a patient, the elongate instrument including an elongate body and a support member disposed within the elongate body. The support member may include a plurality of coils wherein the plurality of coils may include a first coil and a second coil wound in opposite directions. At least a first coil may include a winding with features that overlay or interlay with features of an axially adjacent winding of the first coil. The windings themselves may optionally overlay or interlay. The method may also include advancing the elongate instrument along a pathway in the patient; steering and guiding a distal portion of the elongate instrument toward a target tissue structure through the pathway; transmitting torsion from a proximal end to a distal end of the elongate body, e.g., with no or negligible torsion lag or wind-up while maintaining flexibility of the elongate body; and/or operating an instrument that is operatively coupled to the distal portion of the elongate instrument to diagnose or treat the target tissue structure.

In certain embodiments, the coils may be coaxially arranged. The coils may be configured such that torsion is transmitted with no or negligible torsion lag or wind-up from a proximal end to a distal end of the elongate body and/or to provide torsional stability to the elongate body or instrument. Coils may be wound from a wire'having a cross sectional shape configured to provide overlapping and/or interlocking between axially adjacent windings of a coil. The cross sectional shape of a wire may include various shapes, such as a step shape, parallelogram shape, trapezoidal shape, and T-shape. A coil may be wound from a wire having a cross sectional shape configured to provide overlap between axially adjacent windings of that coil where the overlap may substantially obstruct spacing between axially adjacent windings of a coil such that overlap between radially adjacent coils is negligible or eliminated.

The distance between axially adjacent windings of a coil may vary along a length of the coil such that bending of the elongate instrument or body can be maximized or minimized in different portions of the elongate instrument or body. In certain embodiments a tri-coil configuration is provided. The tri-coil may be configured to provide torsional stability to the elongate body, e.g., by transmitting torsion with no or negligible torsion lag or wind-up from a proximal end to a distal end of the elongate body in two or more rotational directions. Optionally, the axially adjacent windings of a coil may link together to form a solid tube that allows for axial compression and expansion of the elongate body. Optionally, a support member may include coupled or interlocking segments.

In certain embodiments, a steerable elongate instrument is provided which has an inner, middle, and outer coil, wherein the middle coil is wound in the opposite direction as the inner and outer coils such that when a rotational force is applied to the elongate body the middle coil interferes with the inner or outer coils and opposes radial expansion and/or contraction of the inner or outer coil. Optionally, the inner and outer coils may be constructed from a substantially flat wire and the middle coil may be constructed from a round wire or vice versa. A lumen may be provided within the inner coil or the middle or outer coils. Spacing between axially adjacent windings of a coil may vary, e.g., it may range from about 0.00010 to 0.00045 inches and the elongate body has a bend radius that varies, e.g., the bend radius may range from about 7 mm to 12 mm.

In certain embodiments, a steerable elongate instrument is provided. The steerable elongate instrument may include an elongate body and one or more control elements coupled to the elongate body. The control elements may be configured to steer or articulate one or more portions of the elongate body. A support member may be disposed within the elongate body. The support member may be configured to support steering or articulation movements of the elongate body and eliminate, minimize, or reduce rotational or torsional lag or wind-up.

In certain embodiments, the support member may include a plurality of coils. At least two of the plurality of coils of the support member may have opposing coil windings. Windings of at least one of the coils may have features that overlay or interlay with adjacent windings.

Optionally, the support member may be a tubular structure with features or patterns removed from various portions of the tubular structure. Optionally, the support member may be comprised of coupled or interlocking segments and the segments may include features that allow movement between adjacent segments. Optionally, the segments may be coupled together through a spacer member.

In certain embodiments, a method of performing a minimally invasive surgical procedure is provided. The method may include the following steps: inserting an elongate instrument into a patient through an incision or orifice, where the elongate instrument includes a support member that allows at least one degree of freedom of movement of various portions of the elongate instrument; advancing the elongate instrument along a pathway in the patient; steering and guiding a distal portion of the elongate instrument toward a target tissue structure through the pathway; and operating an instrument that is operatively coupled to the distal portion of the elongate instrument to diagnose or treat the target tissue structure.

In certain embodiments, the support member may include a plurality of coils. At least two of the plurality of coils of the support member may have opposing coil windings. Windings of at least one of the coils may have features that overlay or interlay with adjacent windings.

Optionally, the support member may be a tubular structure with features or patterns removed from various portions of the tubular structure. Optionally, the support member may be comprised of coupled or interlocking segments and the segments may include features that allow movement between adjacent segments. Optionally, the segments may be coupled together through a spacer member.

Multiple embodiments and variations have been disclosed and described herein. Many combinations and permutations of the disclosed system may be useful in minimally invasive medical intervention and diagnostic procedures, and the system may be configured to support various flexible robotic instruments. One of ordinary skill in the art having the benefit of this disclosure would appreciate that the foregoing illustrated and described embodiments may be modified or altered, and it should be understood that the embodiments described herein, are not limited to the particular forms or methods disclosed, but also cover all modifications, equivalents and alternatives. Further, the various features and aspects of the illustrated embodiments may be incorporated into other embodiments, even if not so described herein, as will be apparent to those ordinary skilled in the art having the benefit of this disclosure. Although particular embodiments have been shown and described, it should be understood that the above discussion is not intended to be limited to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

1.-23. (canceled)

24. A flexible elongate body comprising:
a plurality of axially extending members;
a support member wherein the support member is configured to provide torsional stability to the flexible elongate body;
a base member;
an end member;
one or more intermediate spacer members;
wherein one or more of the plurality of axial extending members is secured to each of the base member, the end member and at least one of the intermediate spacer members; and
wherein the other of the plurality of axial extending members is secured to the end member and slidably disposed through apertures in at least one of the intermediate spacer members and the base member.

25. The flexible elongate body of claim 24, wherein the support member is positioned along a length of at least one of the axially extending members.

26. The flexible elongate body of claim 24, wherein the support member is configured to surround at least one of the axially extending members.

27. The flexible elongate body of claim 24, wherein the support member comprises a plurality of coaxially arranged helical members wherein the plurality of helical members comprise first and second helical members wound in opposing directions, wherein the first and second helical members are configured such that when a rotational force is applied to the flexible elongate body the first and second helical members are driven in opposing radial directions interfering with one another in opposing radial directions.

28. The flexible elongate body of claim 27, wherein the first helical member comprises a first winding with features that overlay or interlay with features of an axially adjacent winding of the first helical member such that the overlaying or interlaying of the axially adjacent windings of the first helical member minimizes overlap between radially adjacent windings of the first and second helical members.

29. The flexible elongate body of claim 26, wherein the first helical member is wound from a wire having a cross sectional shape configured to provide overlapping or interlocking between axially adjacent windings of the first helical member.

30. The flexible elongate body of claim 29, wherein the cross sectional shape of the wire is selected from the group consisting of a step shape, parallelogram shape, trapezoidal shape, and T-shape.

31. The flexible elongate body of claim 26, wherein a distance of spacing between axially adjacent windings of the first helical member varies along a length of the first helical member such that bending of the flexible elongate body can be maximized or minimized along different portions of the device.

32. The flexible elongate body of claim 26, wherein spacing between at least two of the axially adjacent windings of the first helical member ranges from about 0.00010 to 0.00045 inches and the flexible elongate body has a bend radius of about 7 mm to 12 mm.

33. The flexible elongate body of claim 24, wherein the support member is configured to surround the plurality of axially extending members.

34. The flexible elongate body of claim 24, wherein the plurality of axially extending members are arranged so one or more of said plurality of axially extending members are disposed about and parallel to a centrally located one of the plurality of axially extending members.

35. The flexible elongate body of claim 33, wherein there are three secondary axially extending members that are disposed about and parallel to the centrally located axially extending member.

36. The flexible elongate body of claim 24, wherein the plurality of axially extending members are configured and arranged so as to be flexible in bending and stiff in the axial direction so that the axially extending members do not deform when the elongate body is being manipulated.

37. The flexible elongate body of claim 24, wherein each of the plurality of axially extending members are configured to include a lumen, and the lumens are configured to receive an actuating member.

38. The flexible elongate body of claim 24, wherein the plurality of axially extending members are configured and arranged so as to form a continuous flexible backbone system.

39. The flexible elongate body of claim 34, wherein the flexible backbone system is configured and arranged so as to be capable of at least two degrees of freedom.

40. The flexible elongate body of claim 24, wherein the plurality of axially extending members are configured and arranged so as to form a continuous non-extensible flexible backbone system.

41. The flexible elongate body of claim 24, wherein a tool is operably coupled to a first end of the flexible elongate body and an actuation device is operably coupled to a second end of the flexible manipulation device, wherein the actuation device is configured and arranged to cause the flexible elongate body to maneuver the operably coupled tool in one or more directions responsive to outputs of the actuation device.

42. The flexible elongate body of claim 24, wherein torsion is transmitted with no or negligible torsion lag or wind-up from a proximal end to a distal end of the elongate body.

43. A method of performing a minimally invasive diagnostic, surgical or therapeutic techniques comprising: inserting a flexible elongate body into a patient's body, the flexible elongate body comprising a plurality of axially extending members and a support member wherein the support member is configured to provide torsional stability to the flexible elongate body;
steering the elongate body from a first position to a second position in the body;
transmitting torsion from a proximal end to a distal end of the elongate body with no or negligible torsion lag or wind-up while maintaining flexibility of the elongate body; and
operating an instrument that is operatively coupled to a distal portion of the elongate body to diagnose or treat a target tissue structure in the body.

44. The flexible elongate body of claim 43, wherein the support member is configured to surround at least one of the axially extending members.

45. The method of claim 43, wherein the support member comprises a first helical member positioned along a length of an axially extending member, and a second helical member positioned along the length of an axially extending member the method further comprising;
actively driving the first helical member in a first direction; and
actively driving the second helical member in a second direction opposite the first direction such that the first and second helical members interfere with one another in opposing radial directions to provide torsional stability to the elongate body.

46. The method of claim 45, further comprising allowing overlay or interlay between features of axially adjacent windings of the first helical member to minimize overlap between radially adjacent windings of the first and second helical members.

47. The method of claim 45, wherein the first helical member is wound from a wire having a cross sectional shape configured to provide overlapping or interlocking between axially adjacent windings of the first helical member.

48. The method of claim 47, wherein the cross sectional shape of the wire is selected from the group consisting of a step shape, parallelogram shape, trapezoidal shape, and T-shape.

49. The method of claim 45, wherein a distance of spacing between axially adjacent windings of the first helical member varies along a length of the first helical member such that bending of the flexible elongate body can be maximized or minimized along different portions of the device.

50. The method of claim 45, wherein spacing between axially adjacent windings of the first helical member ranges from about 0.00010 to 0.00045 inches and the flexible elongate body has a bend radius of about 7 mm to 12 mm.

51. The method of claim 43, wherein the plurality of axially extending members are arranged so one or more of the plurality of axially extending members are disposed about and parallel to a centrally located one of the plurality of axially extending members.

52. The method of claim 43, wherein one or more of the plurality of axial extending members is secured to each of a base member, an end member and at least one intermediate spacer member; and
wherein the other of the plurality of axial extending members is secured to the end member and slidably disposed in through apertures in at least one of the intermediate spacer members and the base member.

53. The method of claim 43, wherein the plurality of axially extending members are configured and arranged so as to be flexible in bending and stiff in the axial direction so that the axially extending members do not deform when the elongate body is being manipulated.

54. The method of claim 43, wherein each of the plurality of axially extending members are configured to include a lumen, and the lumens are configured to receive an actuating member.

55. The method of claim 43, wherein the plurality of axially extending members are configured and arranged so as to form a continuous flexible backbone system.

56. The method of claim 55, wherein the flexible backbone system is configured and arranged so as to be capable of at least two degrees of freedom.

57. The method of claim 43, wherein the plurality of axially extending members are configured and arranged so as to form a continuous non-extensible flexible backbone system.

58. The method of claim 43, wherein a tool is operably coupled to a first end of the flexible elongate body and an actuation device is operably coupled to a second end of the flexible elongate body, wherein the actuation device is configured and arranged to cause the flexible elongate body to maneuver the operably coupled tool in one or more directions responsive to outputs of the actuation device.

59. The flexible elongate body of claim 43, wherein the support member is configured to surround the plurality of axially extending members.
